(19) United States
(12) Patent Application Publication
(10) Pub. No.: US 2013/0122297 A1
(43) Pub. Date: May 16, 2013

(54) MOLECULAR GAUGE BLOCKS FOR BUILDING ON THE NANOSCALE

(71) Applicant: NORTHWESTERN UNIVERSITY, Evanston, IL (US)

(72) Inventors: Sergio Grunder, Chicago, IL (US); Adam C. Whalley, Chicago, IL (US); Cory Valente, Yardley, PA (US); J. Fraser Stoddart, Evanston, IL (US)

(73) Assignee: NORTHWESTERN UNIVERSITY, Evanston, IL (US)

(21) Appl. No.: 13/658,973

(22) Filed: Oct. 24, 2012

Related U.S. Application Data
(60) Provisional application No. 61/550,748, filed on Oct. 24, 2011.

(51) Int. Cl. C07C 245/10; C07C 63/331; C07C 65/105

(52) U.S. Cl. CPC C07C 245/10; C07C 65/105; C07C 63/331
USPC 428/401; 562/469; 562/492; 534/660; 534/853; 252/182.12; 252/182.31

(57) ABSTRACT

Disclosed herein is a way to produce a series of discrete sized slender, rigid oligoparaxylene molecules ranging from 1-5 nm in length. Molecules, based on 1-7, 9-11 paraxylene rings, have been synthesized as part of a homologous series of oligoparaxylenes (OPXs) with a view to providing a molecular tool box for the construction of nano architectures—such as spheres, cages, capsules, metal-organic frameworks (MOFs), metal-organic polyhedrons (MOPs) and covalent-organic frameworks (COFs), to name but a few—of well-defined sizes and shapes. Twisting between the planes of contiguous paraxylene rings is generated by the steric hindrance associated with the methyl groups and leads to the existence of soluble molecular gauge blocks without the need—at least in the case of the lower homologues—to introduce long aliphatic side chains onto the phenylene rings in the molecules.

### CROSS REFERENCE TO RELATED APPLICATIONS

The benefit under 35 U.S.C. §119 of U.S. Provisional Application No. 61/550,748, filed Oct. 24, 2011 is claimed, the disclosure of which is incorporated by reference in its entirety.

### BACKGROUND

The technical and financial limitations surrounding fabrication of integrated systems by top-down approaches at the lower end of the nanoscale regime are conspiring together to increase the importance of developing bottom-down approaches to contracting either by strict self-assembly or by template-directed synthesis (itself dependent upon the operation of both molecular recognition and self-assembly processes), molecular building blocks at the higher end of the Angstrom scale.

To be able to control the size and shape of nanoscale architechtures built in three dimensional space (e.g., spheres, cages, capsules, metal-organic frameworks (MOF), metal-organic polyhedrons (MOP), covalent-organic frameworks (COF)) would benefit from creation of modular building blocks for construction on the nanoscale that are similar in concept to the gauge blocks used by engineers working in the macroscopic world. In the interest of creating spaces inside cages and capsules, and porosity within the extended structures of MOFs and COFs, it is important to identify linkers which are slender, rigid, and soluble. Provided herein are such materials.

### SUMMARY

Disclosed herein are compounds useful as nanoscale molecular gauge blocks. More specifically, provided herein are compounds having a structure of formula (I):

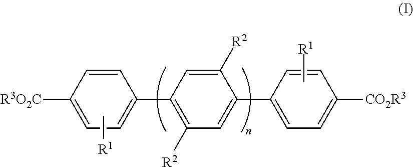

wherein each R¹ is independently H, OH, alkyl, alkoxy, or amino; each R² is independently C₁-C₁₂ alkyl, C₁-C₁₂ haloalkyl, nitro, amino, CHO, alkyleneCHO, CN, alkoxy, halo, alkyleneferrocene, —N═NH-aryl, or polyalkyleneoxide; each R³ is independently H, alkyl, or alkylenearyl, and n is an integer of 1 to 15.

Specific compound disclosed as molecular gauge blocks contemplated include

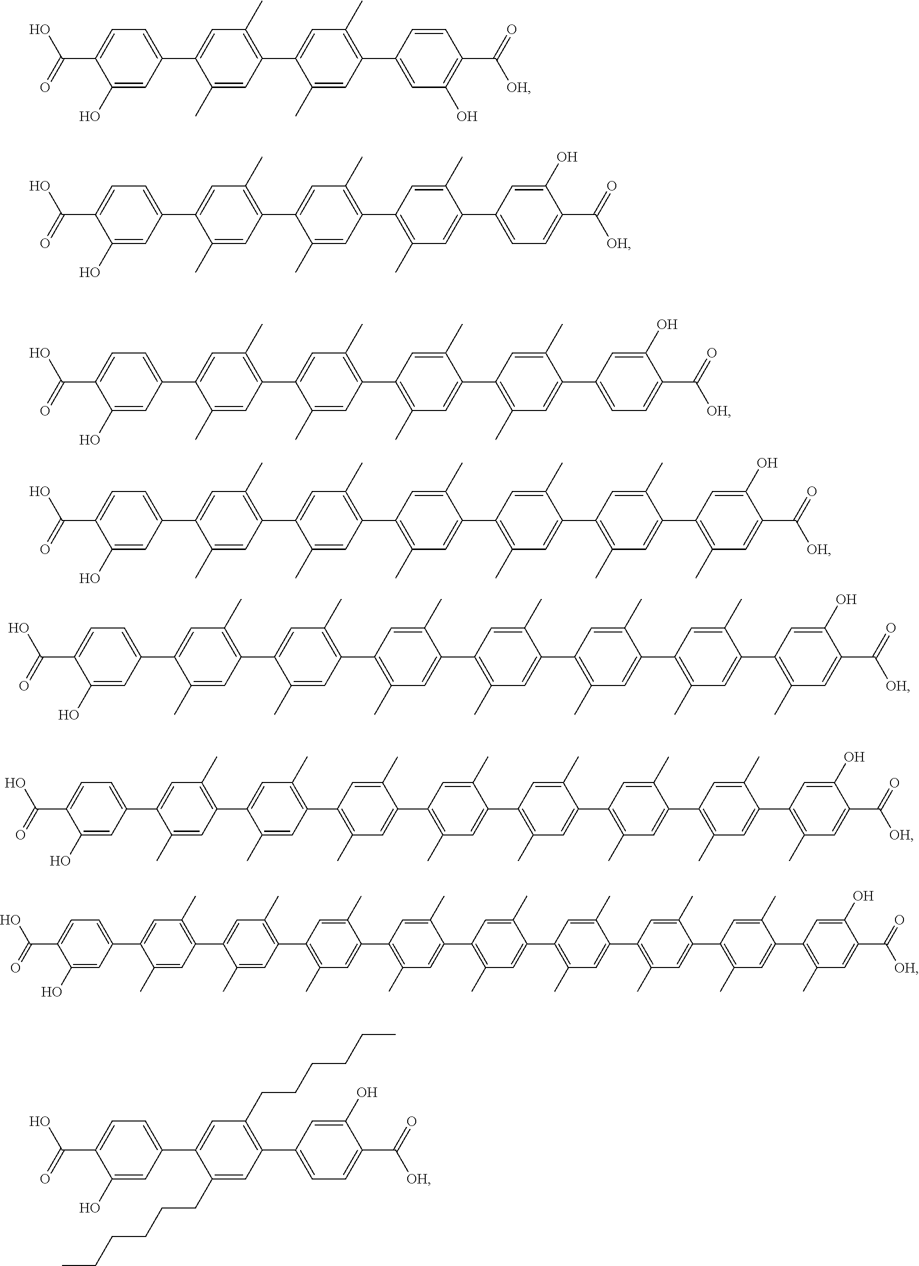

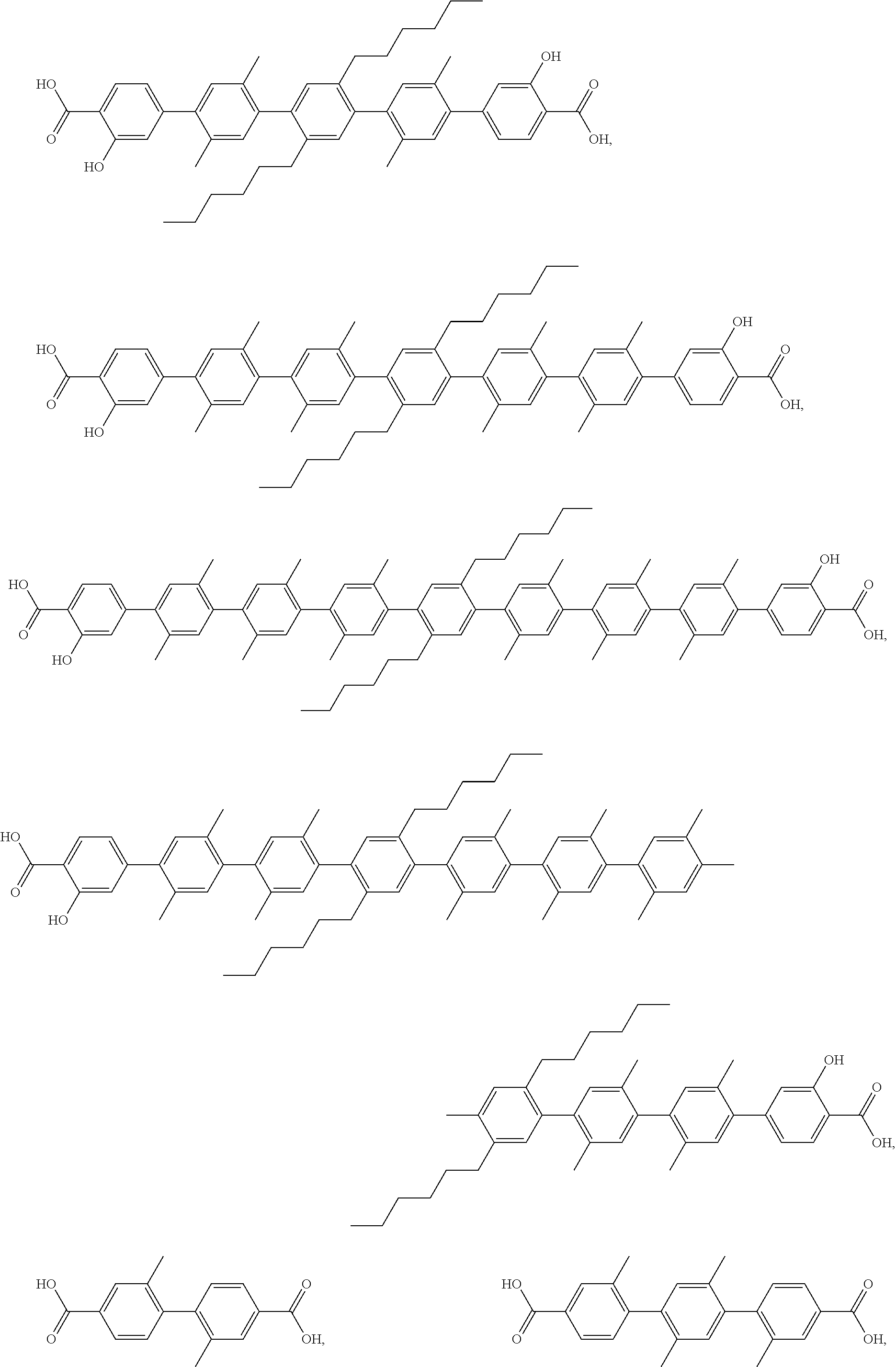

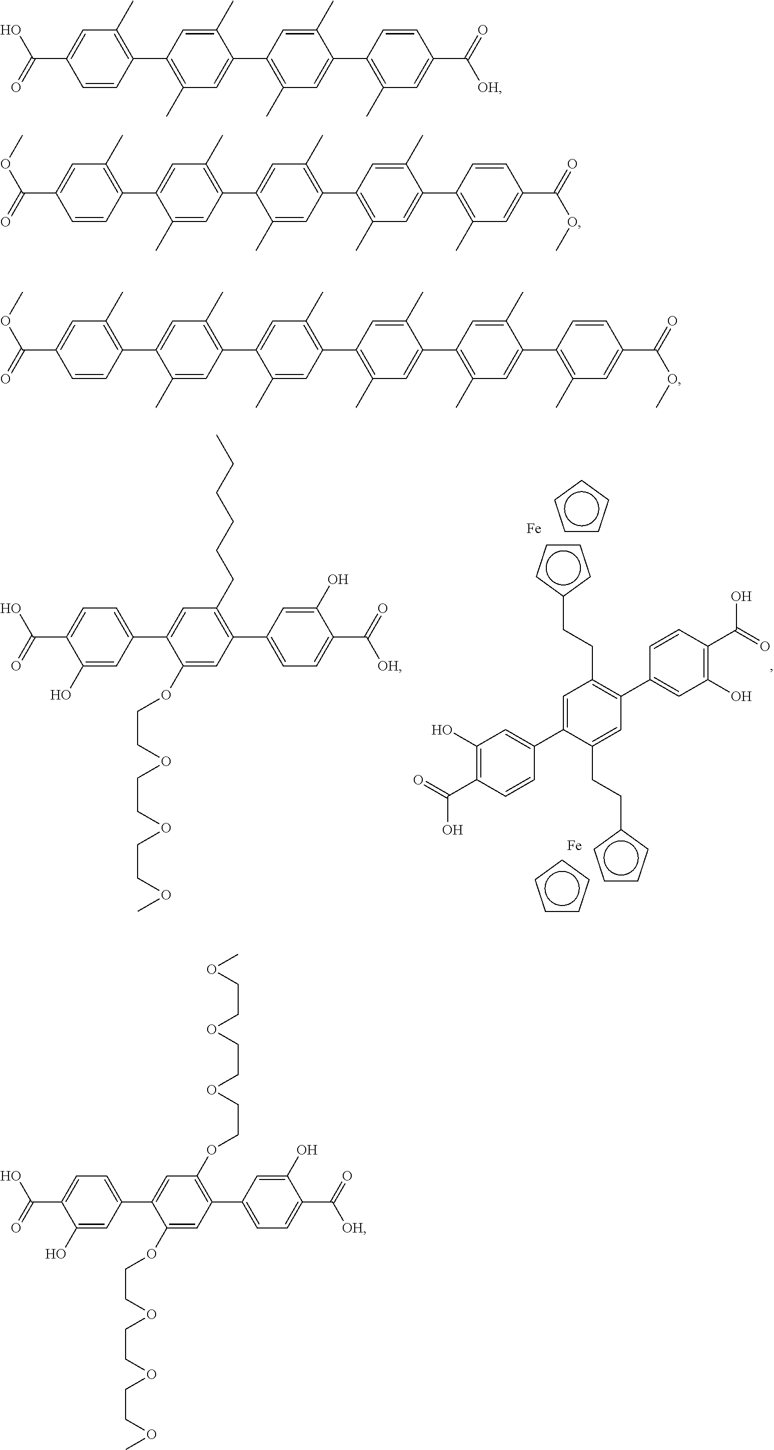

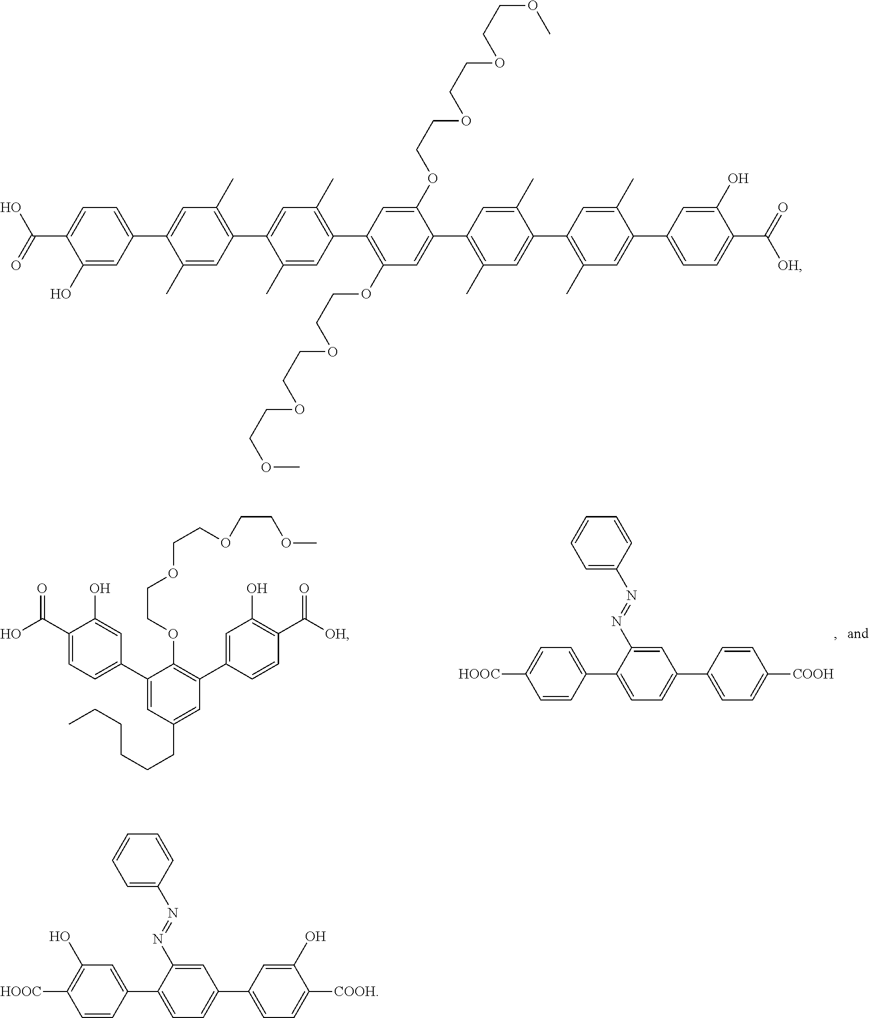 [SMILES: COCCOCCOCCOc1cc(-c2cc(C)c(-c3cc(C)c(-c4ccc(C(=O)O)c(O)c4)cc3C)cc2C)c(OCCOCCOCCOC)cc1-c1cc(C)c(-c2cc(C)c(-c3ccc(C(=O)O)c(O)c3)cc2C)cc1C ; CCCCCCc1cc(-c2ccc(C(=O)O)c(O)c2)c(OCCOCCOCCOC)c(-c2ccc(C(=O)O)c(O)c2)c1 ; O=C(O)c1ccc(-c2ccc(-c3ccc(C(=O)O)cc3)c(/N=N/c3ccccc3)c2)cc1 ; O=C(O)c1ccc(-c2ccc(-c3ccc(C(=O)O)c(O)c3)c(/N=N/c3ccccc3)c2)cc1O]

Further provided herein are nanofibers comprising the compounds disclosed herein. In addition, provided herein are micro-spheres comprising the compounds disclosed herein. Also provided are gels comprising the compounds disclosed herein.

### BRIEF DESCRIPTION OF FIGURES

FIG. 1. SEM images of A) hex-3-mer-HCA, B) hex-5-mer-HCA, C) hex-7-mer-HCA. All samples were dropcasted from Me2SO on silicon wafer. D) photographs of hex-7-mer-HCA in solution (left) and gel (right) state (10 mM in Me₂SO).

FIG. 2: a) (R)-2-Mer-ME and its minor image (S)-2-mer-ME. Both compounds have a C2 rotation axis of symmetry which is perpendicular to the phenylene-connecting bond and an angle which is half the angle formed by the two planes of the phenylenes. b) Having three rotationally hindered phenylenes leads to three atropisomers. (RR)-3-Mer-ME and (SS)-3-mer-ME are chiral and have a C2 axis of rotation. (RS)-3-mer-ME is achiral with a point of inversion as a symmetry element. c) The atropisomers with four hindered phenylenes exist as three pairs of enantiomers, two of it (RRR/SSS & RSR/SRS) with a C2 axis of rotation, one asymmetric pair (RRS/SSR). VT NMR experiments of compound 3-mer-ME (d) and 4-mer-ME (e) in toluene-d8. The spectra were taken in 10 K increments and the system was allowed to equilibrate at every temperature for 15 minutes. On the NMR time scale all of the expected methyl peaks are observed, resulting in a total number of methyl peaks of four for compound 3-mer-ME and 12 for compound 4-mer-ME.

FIG. 3: Modeled structures in a relative length scale of the compounds consisting of one up to eleven phenylene rings. The hydrogen atoms and hexyl groups are omitted for clarity.

### DETAILED DESCRIPTION

Molecular gauge blocks, based on 1-7, 9-11 paraxylene rings, have been synthesized as part of a homologous series of oligoparaxylenes (OPXs) with a view to providing a molecular tool box for the construction of nano architectures—such as spheres, cages, capsules, metal-organic frameworks (MOFs), metal-organic polyhedrons (MOPs) and covalent-organic frameworks (COFs), to name but a few—of well-defined sizes and shapes. Twisting between the planes of contiguous paraxylene rings is generated by the steric hindrance associated with the methyl groups and leads to the existence of soluble molecular gauge blocks without the need—at least in the case of the lower homologues—to introduce long aliphatic side chains onto the phenylene rings in the molecules. Although soluble molecular gauge blocks with up to seven consecutive benzenoid rings have been prepared employing repeating para-xylene units, in the case of the higher homologues, it becomes necessary to introduce hexyl groups instead of methyl groups onto selected phenylene rings to maintain solubility. A hexyl-doped compound with seven substituted phenylene rings was found to be an organogelator, exhibiting thermally reversible gelation and a critical gelation concentration of 10 mM in dimethyl sulfoxide. Furthermore, control over the morphology of a series of hexyl-doped OPXs to give microfibers, microaggregates, or nanofibers, was observed as a function of their lengths according to images obtained by scanning electron microscopy. The modular syntheses of the paraphenylene derivatives rely heavily on Suzuki-Miyaura cross-coupling reactions. The lack of π-π conjugation in these derivatives that is responsible for their enhanced solubilities was corroborated by UV/Vis and fluorescent spectroscopies. In one particular series of model OPXs, dynamic ¹H NMR spectroscopy was used to probe the stereochemical consequences of having from one up to five axes of chirality present in the same molecule. The Losanitsch sequence for the compounds with 1-3 chiral axes was established, and a contemporary mathematical way was found to describe the sequence.

Molecular gauge blocks are disclosed herein that have a structure of formula (I):

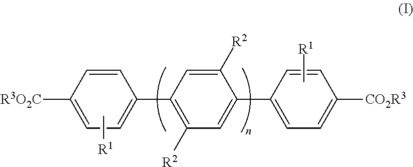

wherein each R¹ is independently H, OH, alkyl, alkoxy, or amino; each R² is independently C₁-C₁₂ alkyl, C₁-C₁₂ haloalkyl, nitro, amino, CHO, alkyleneCHO, CN, alkoxy, halo, alkyleneferrocene, —N═NH-aryl, or polyalkyleneoxide; each R³ is independently H, alkyl, or alkylenearyl, and n is an integer of 1 to 15.

The term “alkyl” used herein refers to a saturated or unsaturated straight or branched chain hydrocarbon group of one to forty carbon atoms, including, but not limited to, methyl, ethyl, n-propyl, isopropyl, n-butyl, isobutyl, tert-butyl, n-hexyl, and the like. Alkyls of one to six carbon atoms are also contemplated. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halide, thiol (SH), aryl, heteroaryl, cycloalkyl, heterocycloalkyl, and amino. It is specifically contemplated that in the compounds described herein the alkyl group consists of 1-40 carbon atoms, preferably 1-25 carbon atoms, preferably 1-15 carbon atoms, preferably 1-12 carbon atoms, preferably 1-10 carbon atoms, preferably 1-8 carbon atoms, and preferably 1-6 carbon atoms.

The term “alkoxy” used herein refers to straight or branched chain alkyl group covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “haloalkyl” used herein refers to straight or branched chain alkyl group substituted with one or more halo atoms (e.g., F, Cl, Br, and/or I). Non-limiting examples of haloalkyl groups include CF₃, CH₂CF₃, CCl₃, and the like.

The term “alkylene” used herein refers to an alkyl group having a substituent. For example, the term “alkylene aryl” refers to an alkyl group substituted with an aryl group. The alkylene group is optionally substituted with one or more substituent previously listed as an optional alkyl substituent. For example, an alkylene group can be —CH₂CH₂— or —CH₂—.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

The term “amino” as used herein refers to —NR₂, where R is independently hydrogen, optionally substituted alkyl, optionally substituted heteroalkyl, optionally substituted cycloalkyl, optionally substituted heterocycloalkyl, optionally substituted aryl or optionally substituted heteroaryl. Non-limiting examples of amino groups include NH₂, NH(CH₃), and N(CH₃)₂. In some cases, R is independently hydrogen or alkyl.

As used herein, the term “polyalkyleneoxide” refers to a group having repeating alkylene groups separated by a —O—, the alkylene can be, e.g., a C₁-C₄alkylene, such as an ethylene, methylene, propylene, or the like. An example of a contemplated polyalkylene oxide is —O(CH₂CH₂O)ₘCH₃, where m is 1 to 20, or more specifically 3 to 10.

The development of the ways and means to make molecular gauge building blocks will have positive re-percussions on the control of nano-structures in general. Their in-corporation into extended structures with the MOF-74 topology provides an excellent demonstration of the potential usefulness of these molecular gauge blocks.

The technical and financial limitations surrounding the fabrication of integrated systems by top-down approaches at the lower end of the nanoscale regime are conspiring together to increase the importance of developing bottom-up approaches to constructing either by strict self-assembly[1] or by template-directed synthesis[2]—itself dependent on the operation of both molecular recognition[3] and self-assembly processes[4]—molecular building blocks at the higher end of the Ångstrom scale.

For we as chemists to be able to control the size and shape of nanoscale architectures built in three-dimensional space—be they spheres[5], cages[6] and capsules[7] or metal-organic frameworks (MOFs)[8], metal-organic polyhedrons (MOPs)[9], covalent-organic frameworks (COFs)[10], or other artificial architectures[4a, 11]—it is useful to evolve a chemistry capable of providing modular building blocks for construction[12] on the nanoscale that are similar in concept to the gauge blocks used by engineers working in the macroscopic world. In the interests of creating space inside cages and capsules, and porosity within the extended structures of MOFs and COFs, it is useful to identify linkers which are slender, rigid and soluble, at least at the beginning of the construction phase.

At the molecular level, possible structural linkers are the oligophenylenes[13] except that, once they are longer than a couple of phenylene units, the compounds are plagued by low solubilities.[14] Provided herein are ways to overcome the characteristic low solubilities of the oligophenylenes by exploring the chemistry of the related oligoparaxylenes (OPXs). It transpires that, in the case of these OPXs, the steric hindrance experienced between the methyl groups located on contiguous benzenoid rings induces a twist in the planes of the paraxylene rings that results in the breaking of the π-π conjugation[15], as well as the disruption of the intermolecular π•••π stacking leading to a dramatic increase in their solubilities. The OPXs have already been recognized as rigid and non-conjugated gauges for charge transfer investigations.[14-16]

Provided herein are (i) the design criteria and synthetic strategy for building soluble, slender, and rigid organic gauge blocks, (ii) the detailed synthesis of all the OPX struts, (iii) the control over morphology as a function of the length on the hexyl-doped OPX series, (iv) stereochemical investigation of the atropisomers with one up to as far as five axes of chirality and a mathematical description of the Losanitsch sequence and, as an example of an application, (v) the use of those blocks for defining the size of pore apertures in metal-organic frameworks.

### Results and Discussion

Synthesis: At the outset before tackling the syntheses of a series of palindromic oligophenylenes the following issues are addressed. (1) Substituents and Solubility. The notoriously low solubilities of the parent oligophenylenes are circumvented by introducing pairs of methyl groups with para dispositions onto all but the terminal phenylene rings in order to impose torsional twists and hence nonplanarity between contiguous aromatic rings. This level of stereoelectronic control reduces the π-π conjugation between the rings and also impairs π•••π stacking between the OPX molecules, leading to the enhanced solubilities the OPXs show compared with their oligophenylene counterparts. Ultimately, some of the methyl groups were replaced on selected OPX rings by hexyl substituents. (2) Functionality and Additional Substituents. For the first homologous series of palindromic OPXs α-hydroxy-carboxylic acids (HCAs) were introduced onto the terminal phenylene rings. The precursors to these derivatives were benzyloxy-methyl esters (BMEs). Subsequently, we also synthesized a homologous series of palindromic OPXs substituted on their terminal phenylene rings with methyl esters (ME) and methyl groups oriented meta to each other. This suite of OPXs was employed to probe the consequences of introducing progressively more and more axes of chirality into the OPXs. (3) Quantities. The methods of syntheses of the OPXs were selected such that they could be accessed in several gram quantities with high purities. In order to accomplish this goal, the gauge block methyl 2-(benzyloxy)-4-(pinacolboronic ester) benzoate (3) on a 100 g scale was synthesized, starting from the commercially available methyl 4-iodosalicylate (1).

With many subsequent Suzuki-Miyaura cross-coupling reactions (Scheme 1) in mind, protection of the hydroxyl groups in the HCAs had to be considered. The benzyl ether proved to be suitable because of (i) its facile introduction and the ease of isolation of the intermediates on a large scale, (ii) its stability towards the coupling conditions and (iii) its straightforward and efficient removal by hydrogenolysis.

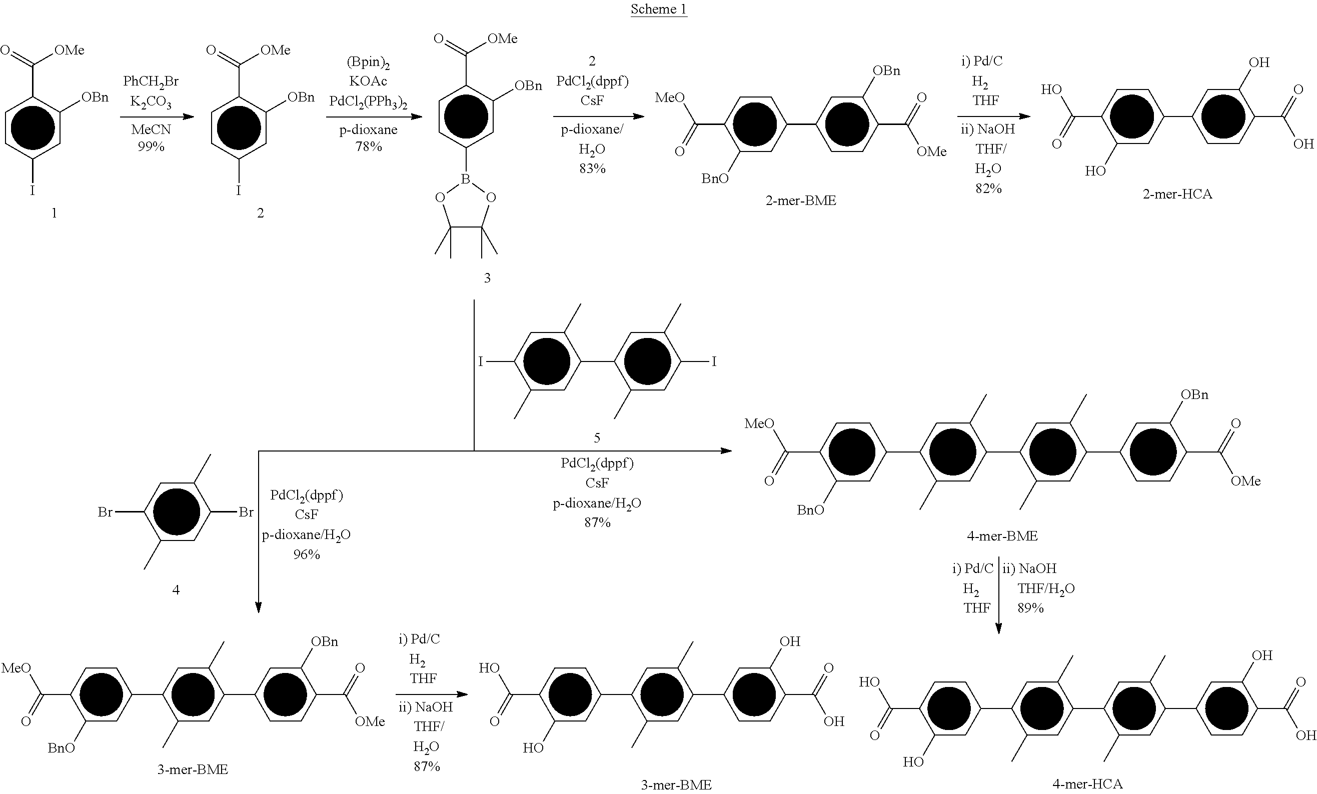

Methyl 4-iodosalicylate (1) was reacted (Scheme 1) with benzyl bromide in MeCN under reflux overnight and, after removal of the insoluble salts by filtration, methyl 2-(benzyloxy)-4-iodobenzoate (2) was obtained as a solid which was pure by 1H NMR spectroscopy. The boronic ester was then introduced using standard Pd-catalyzed chemistry to provide yellow needles of 3 in 78% yield after filtration of the crude reaction mixture through Celite, concentration of the filtrate and recrystallization of the residue.

Suzuki-Miyaura cross-coupling reactions were ideally suited to the assembly of the OPX backbones. Conditions involving PdCl₂(dppf) and CsF in a p-dioxane/H₂O (v/v 2:1) solvent mixture were found to be high yielding, tolerant to the protecting groups and generally applicable throughout the entire series of the OPXs. Applying these conditions, henceforth referred to as the standard Suzuki-Miyaura cross-coupling conditions, to the reaction of the boronic ester building block 3 with the iodide 2 led to the formation of the 2-mer-BME which precipitated out of the reaction mixture and was isolated by filtration in 83% yield. In order to remove the benzyl ether protecting groups, 2-mer-BME was exposed to standard hydrogenolysis conditions of Pd/C in THF under an atmosphere of H2. Thereafter, the methyl esters were saponified in a THF/0.5 M aq NaOH (v/v 1:1) mixture to yield 2-mer-HCA as a colorless solid after acidification of the aqueous layer. Coupling 2.2 equiv of the key building block 3 with 1.0 equiv of the commercially available 2,5-dibromo-paraxylene (4) afforded 3-mer-BME in 96% yield. After hydrogenolysis and saponification, 3-mer-HCA was obtained as a colorless solid in 87% yield over the two reaction steps. In order to obtain 4-mer-HCA, 3 was first of all reacted with 4,4′-diiodo-2,2′,5,5′-tetramethylbiphenyl (5)[15a] yielding 4-mer-BME in 87% yield. Thereafter, 4-mer-BME was subjected to hydrogenolysis and saponification in order to obtain the 4-mer-HCA as a colorless solid in 89% yield over the two reaction steps.

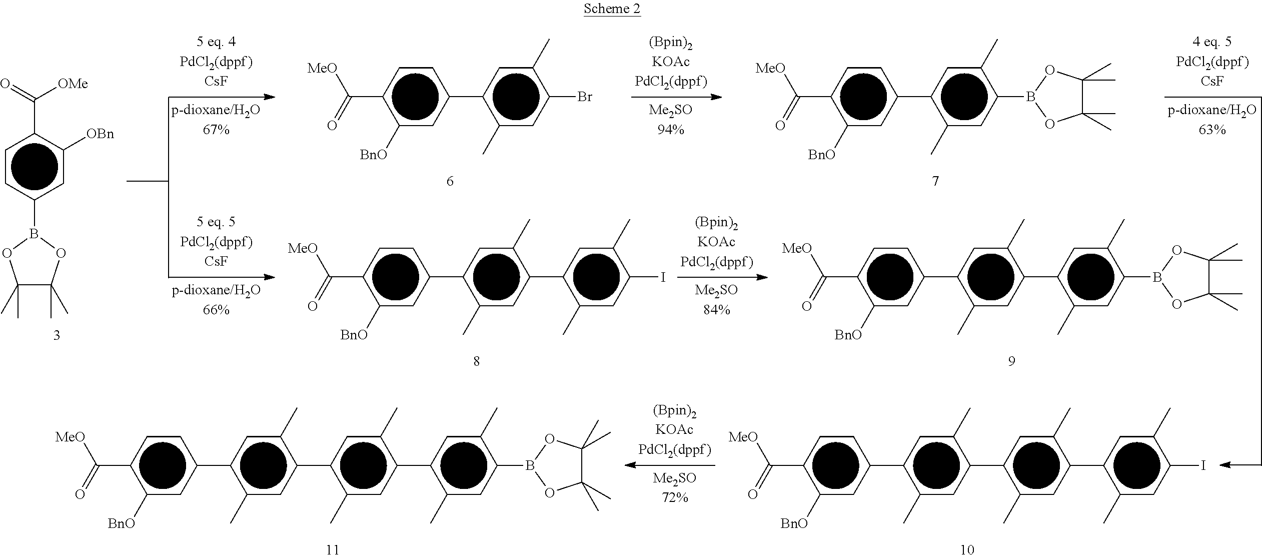

In order to synthesize the higher homologues of the OPXs, a strategy was developed involving the extension of the terminal boronic ester building blocks, bearing carboxyl and hydroxyl groups, before coupling them to an appropriate dihalide to constitute the midriffs of 6-mer-HCA and 7-mer-HCA. The extension of the terminal building block was achieved (Scheme 2) by reacting 3 with an excess of the appropriate dihalide in order to statistically favor the mono-coupled product. Thus, when 3 was reacted using the standard Suzuki-Miyaura conditions with 5.0 equiv of 2,5-dibromo-paraxylene (4), the desired bromide 6 was isolated by flash chromatography in 67% yield. Subsequently, the bromide 6 was converted to the boronic acid pinacol ester 7 in 94% isolated yield employing a Pd-catalyzed cross-coupling reaction. By applying a similar reaction procedure, but by using an excess of 4,4′-diiodo-2,2′,5,5′-tetramethylbiphenyl (5) as the dihalide, the terphenyl derivative 8 was obtained in 66% yield. In order to effect an increase in the number of phenylene units, compound 7 was reacted in another statistically driven Suzuki-Miyaura cross-coupling reaction with an excess of the diiodide 5 to yield (63%) compound 10, which was converted subsequently to the boronic acid pinacol ester to provide building block 11 in 72% yield. With all the boronic ester building blocks in hand, the assemblies of the longer OPX rods (Scheme 3) were tackled.

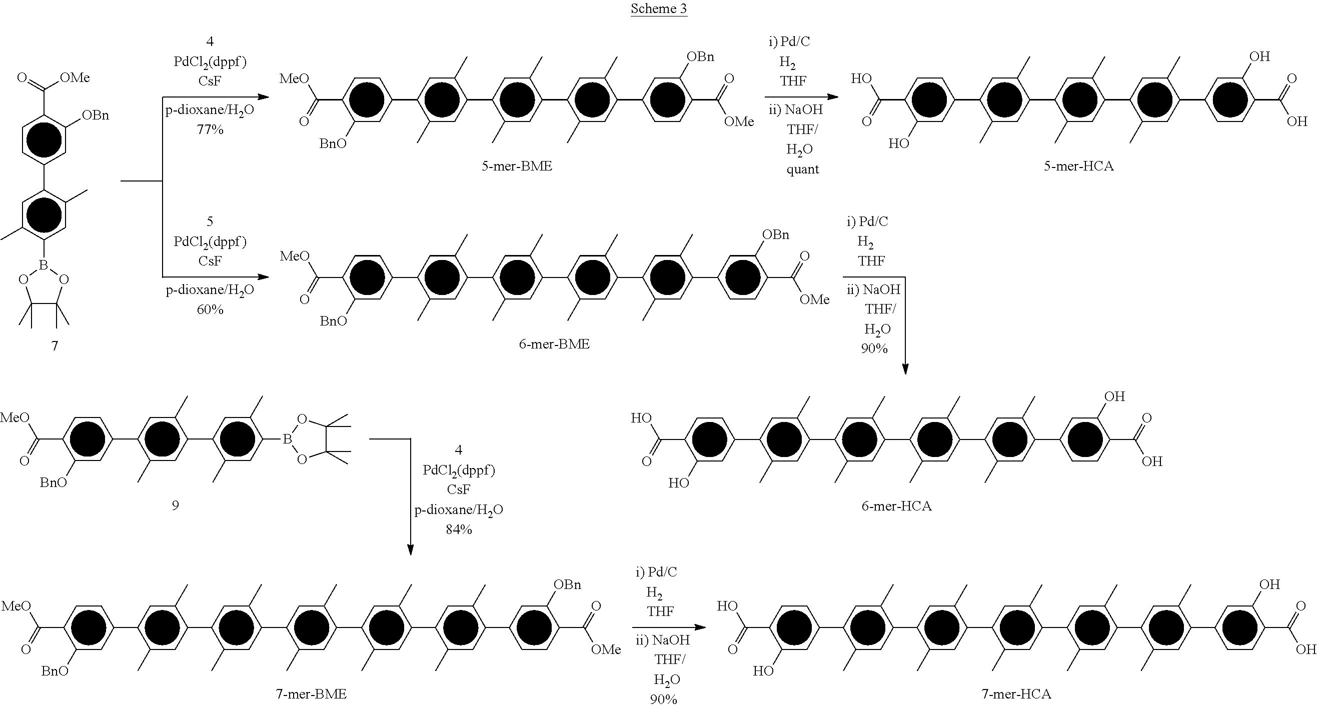

Compound 7 was reacted with 2,5-dibromo-paraxylene (4) using the standard Suzuki-Miyaura cross-coupling conditions to yield 5-mer-BME in 77% yield. After hydrogenolysis and saponification 5-mer-HCA was obtained as a colorless solid. Coupling 7 with compound 5 led to the formation of 6-mer-HCA after hydrogenolysis and saponification of 6-mer-BME. With the objective of synthesizing 7-mer-HCA, 9 was coupled in a Suzuki-Miyaura reaction with 4 yielding (85%) 7-mer-BME which was still easily soluble in common organic solvents. The solubility decreased dramatically, however, after removal of these protecting groups: more than 1000 scans were required to record an acceptable ¹H NMR spectrum on a saturated solution of 7-mer-HCA at 80° C. in CD₃SOCD₃. While it was possible to assemble the backbones of the compounds having eight, nine, and ten contiguous paraxylene units by coupling compounds 9 and 11 with 4 and 5, it was not possible to characterize the corresponding n-mer-HCAs (n=8, 9, 10) employing the normal analytical methods because of their low solubilities in most common solvents. In order to access even longer OPXs, we had to find another way of increasing their solubilities. Hence, we set out to prepare a series of hexyl-doped OPXs in which a few selected methyl groups were replaced by hexyl substituents.

A key building block for these hexyl-doped OPXs—namely 1,4-dihexyl-2,5-diiodobenzene (12)—was prepared in three steps, starting from 1,4-diiodobenzene following a reported procedure.[17] Coupling the boronic ester building blocks 3, 7, 9 and 11 with 12 using the standard Suzuki-Miyaura coupling conditions (Scheme 4) led to the isolation of hex-3-mer-BME, hex-5-mer-BME, hex-7-mer-BME and hex-9-mer-BME in 74-91% yields. Following hydrogenolysis and subsequent saponification, hex-3-mer-HCA, hex-5-mer-HCA, hex-7-mer-HCA and hex-9-mer-HCA were isolated, all as colorless solids, in quantitative yields.

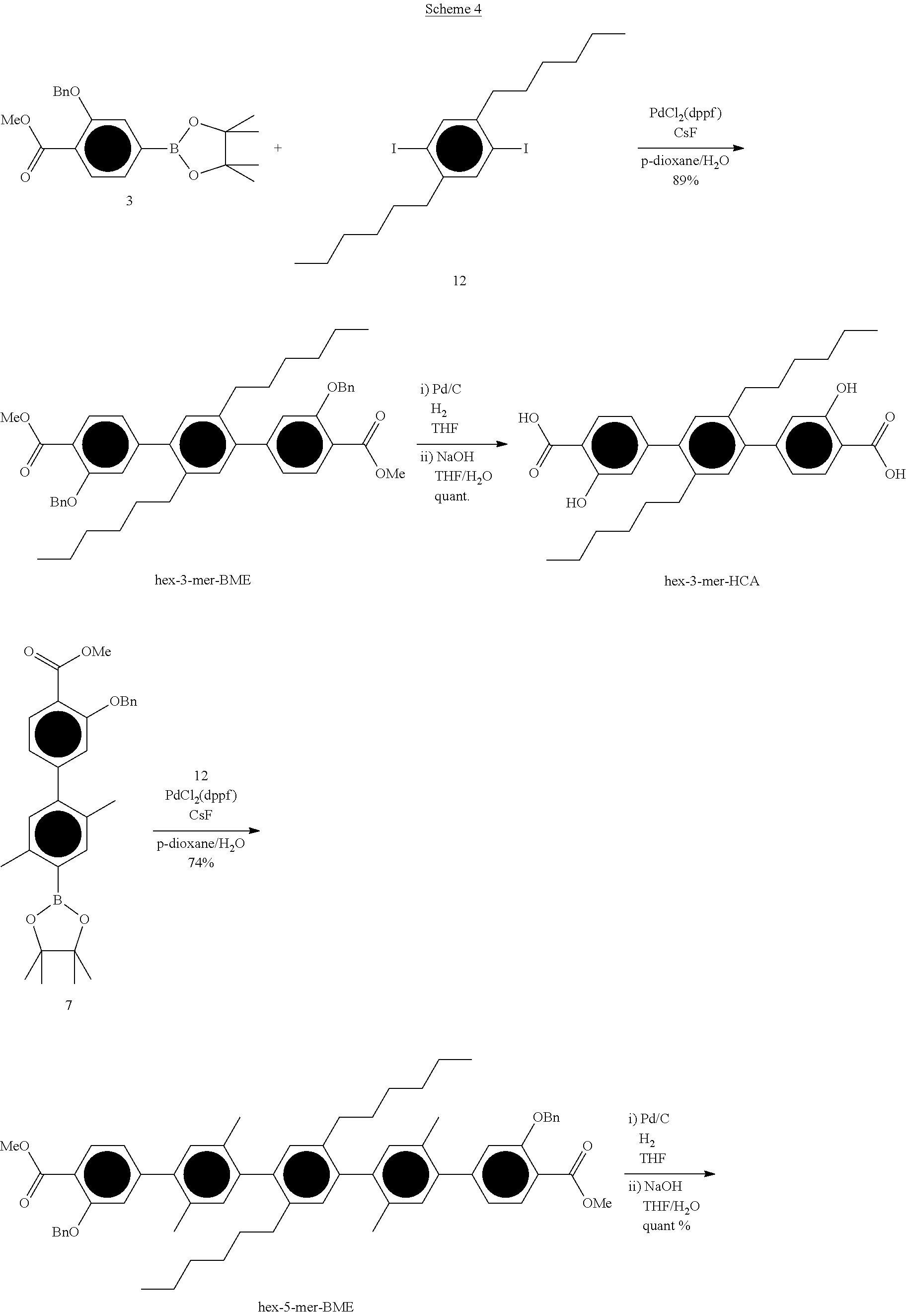 [SMILES: COC(=O)C1CCC(B2OC(C)(C)C(C)(C)O2)CC1OCc1ccccc1 ; CCCCCCC1CC(I)C(CCCCCC)CC1I ; CCCCCCC1CC(C2CCC(C(=O)OC)C(OCc3ccccc3)C2)C(CCCCCC)CC1C1CCC(C(=O)OC)C(OCc2ccccc2)C1 ; CCCCCCC1CC(C2CCC(C(=O)O)C(O)C2)C(CCCCCC)CC1C1CCC(C(=O)O)C(O)C1 ; COC(=O)C1CCC(C2CC(C)C(B3OC(C)(C)C(C)(C)O3)CC2C)CC1OCc1ccccc1 ; CCCCCCC1CC(C2CC(C)C(C3CCC(C(=O)OC)C(OCc4ccccc4)C3)CC2C)C(CCCCCC)CC1C1CC(C)C(C2CCC(C(=O)OC)C(OCc3ccccc3)C2)CC1C]

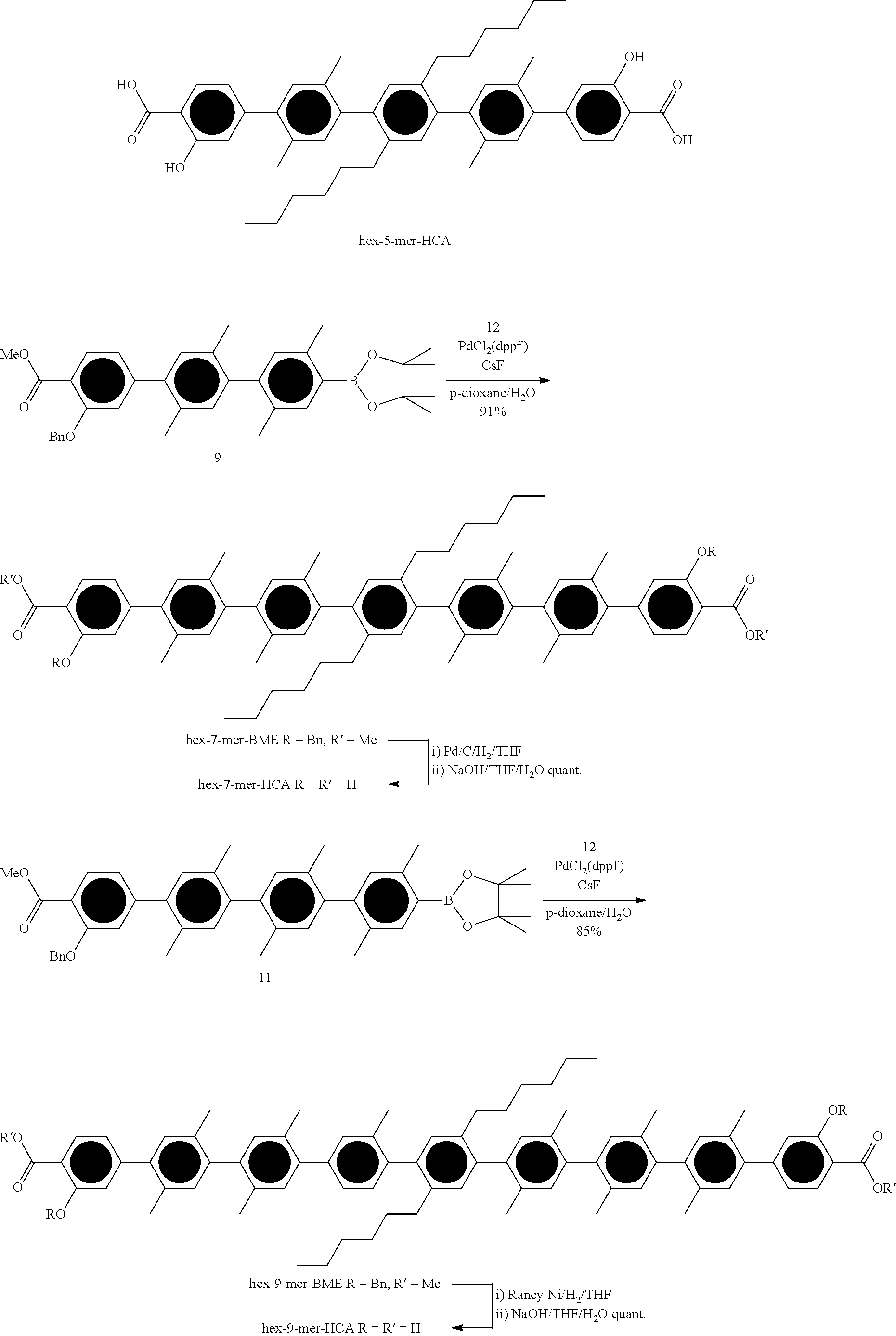

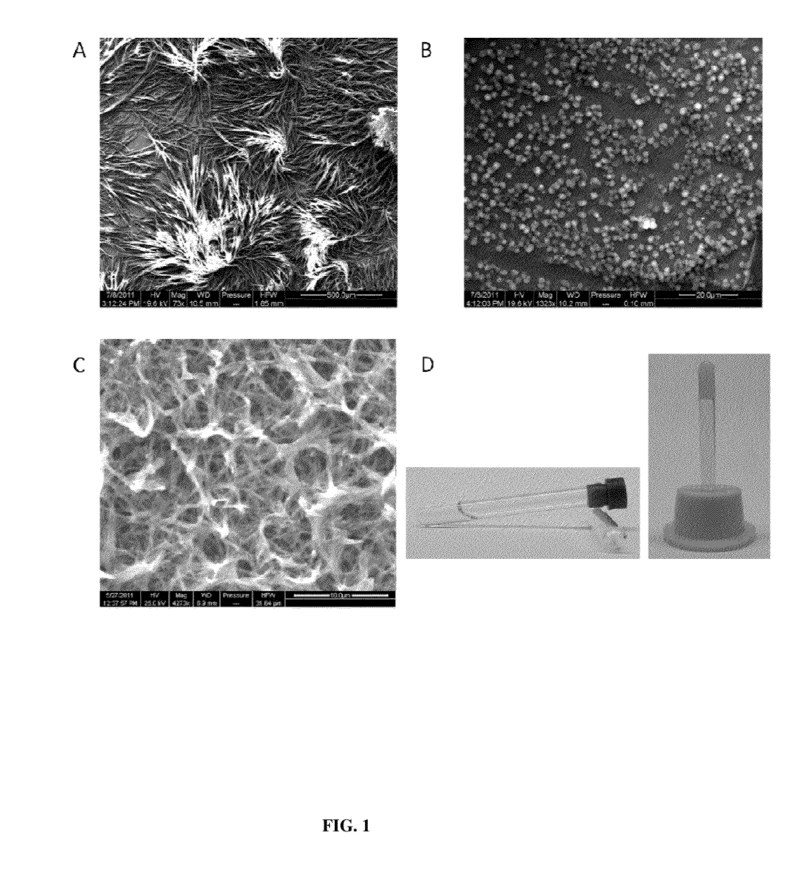

The introduction of hexyl chains onto the central benzenoid ring of the molecular struts not only led to increased solubility, but it also gave rise to some self-assembly-based phenomena. Experiments were conducted on hex-3-mer-HCA, hex-5-mer-HCA and hex-7-mer-HCA in Me2SO. Scanning electron microscopy (SEM) images of the dried sample of hex-3-mer-HCA obtained by drop-casting a Me2SO solution revealed (FIG. 1A) the presence of self-assembled tape-like morphologies. On the other hand, self-assembly of hex-5-mer-HCA resulted (FIG. 1B) in the formation of micro-spheres. The SEM image of hex-7-mer-HCA revealed (FIG. 1C) the presence of intertwined and extended fibrillar network morphologies. These SEM images indicate the stronger self-assembling property of hex-7-mer-HCA when compared with hex-3-mer-HCA and hex-5-mer-HCA. The superior self-assembling properties of hex-7-mer-HCA, in comparison with hex-3-mer-HCA and hex-5-mer-HCA, was also observed in gelation experiments. While hex-3-mer-HCA and hex-5-mer-HCA do not form gels, hex-7-mer-HCA produces an organogel in Me₂SO at concentrations above 10 mM. Photographs of hex-7-mer-HCA in the solution and gel state are displayed in FIG. 1D. Upon heating the gel, the self-assembly breaks and results in the solution state. Self-assembly and gelation can be made to be reversible by cooling the solution to room temperature, demonstrating its thermoreversible characteristics.

Finally, the synthesis of hex-11-mer-HCA (Scheme 5) was investigated with four hexyl substituents. The terphenylboronic acid pinacol ester building block 9 was extended by reaction with an excess of 12 under the standard Suzuki-Miyaura coupling conditions to give the iodide 16 in 40% yield as a colorless solid. In order that it can serve as the central building block, the diboronic ester terphenyl derivative 15 was prepared, starting from 2,5-dibromo-paraxylene (4). Both bromides in compound 4 were substituted with a boronic ester to yield 13 which was subsequently treated with an excess of 4 in a Suzuki-Miyaura reaction in order to favor the statistical formation of the dibromo terphenyl derivative 14. Once again, the bromides were substituted with boronic esters, providing the diboronic ester building block 15 which (as 1.0 equiv) was reacted with 2.2 equiv of the iodide 16, applying the standard Suzuki-Miyaura coupling conditions. Hex-11-mer-BME precipitated out of the reaction mixture and was filtered off after being cooled to room temperature, washed with H₂O and MeOH, and finally purified by column chromatography. After hydrogenolysis (H2/Raney Ni in THF) and subsequent saponification, hex-11-mer-HCA was isolated as a colorless powder.

Since the hindered rotation of the ortho-methyl-substituted oligophenylenes confers elements of axial chirality, it was desirable that we investigate the resulting atropisomers. In order to address this rich stereochemical feature, a series of OPXs were synthesized wherein the ortho-hydroxyl groups were eradicated from both terminal phenylene units, and methyl groups meta to the carboxyl groups were added so as to restrict the relative torsional angles between every single phenylene unit along the backbone of the OPXs. Commercially available methyl-4-bromo-3-methylbenzoate (17) was converted into its boronic ester 18 which was then coupled—using the general Suzuki-Miyaura coupling conditions—with 18, 4 and 5 to provide (Scheme 6) 2-mer-ME, 3-mer-ME and 4-mer-ME in turn. In order to obtain higher homologues, 18 was extended by a further phenylene unit by reacting it with an excess of dibromide 4 and subsequently substituting the bromide with a pinacol boronic ester, leading to compound 20 which was coupled—using the general Suzuki-Miyaura coupling conditions—with 4 and 5, providing 5-mer-ME and 6-mer-ME, respectively.

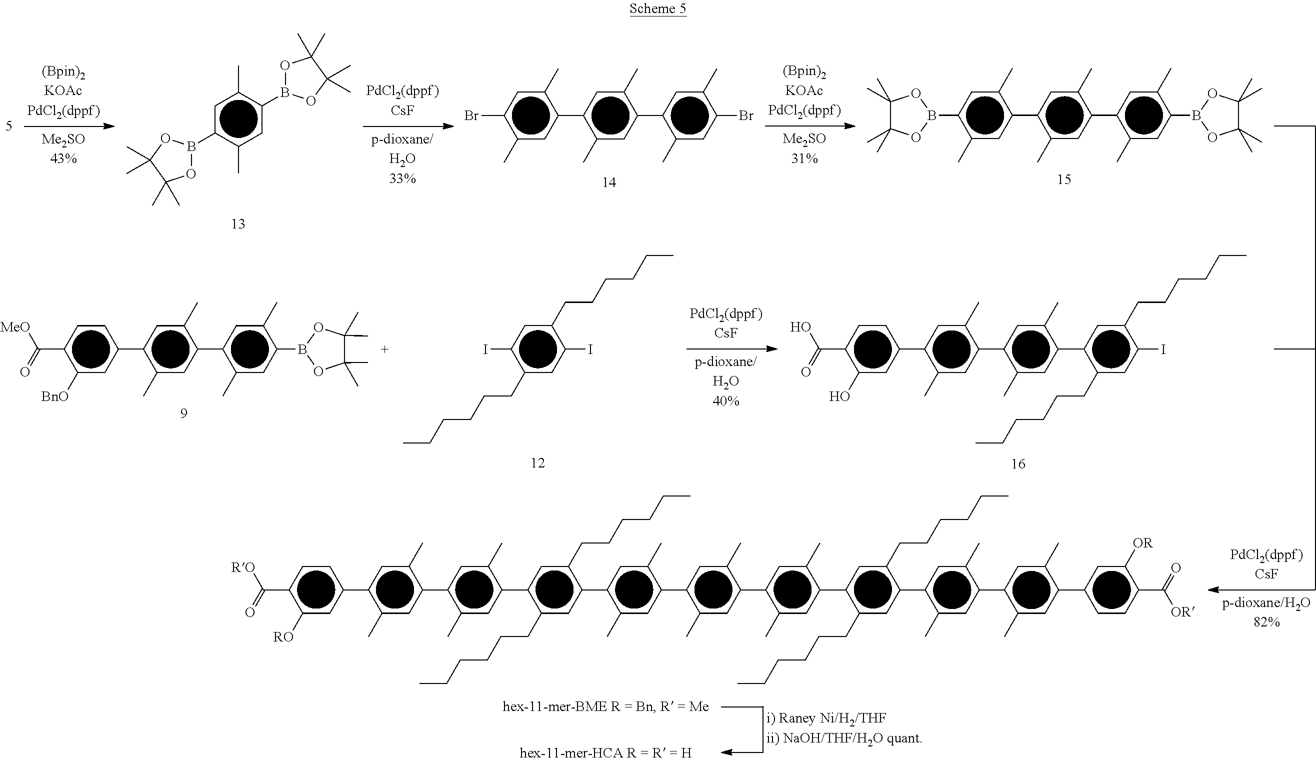

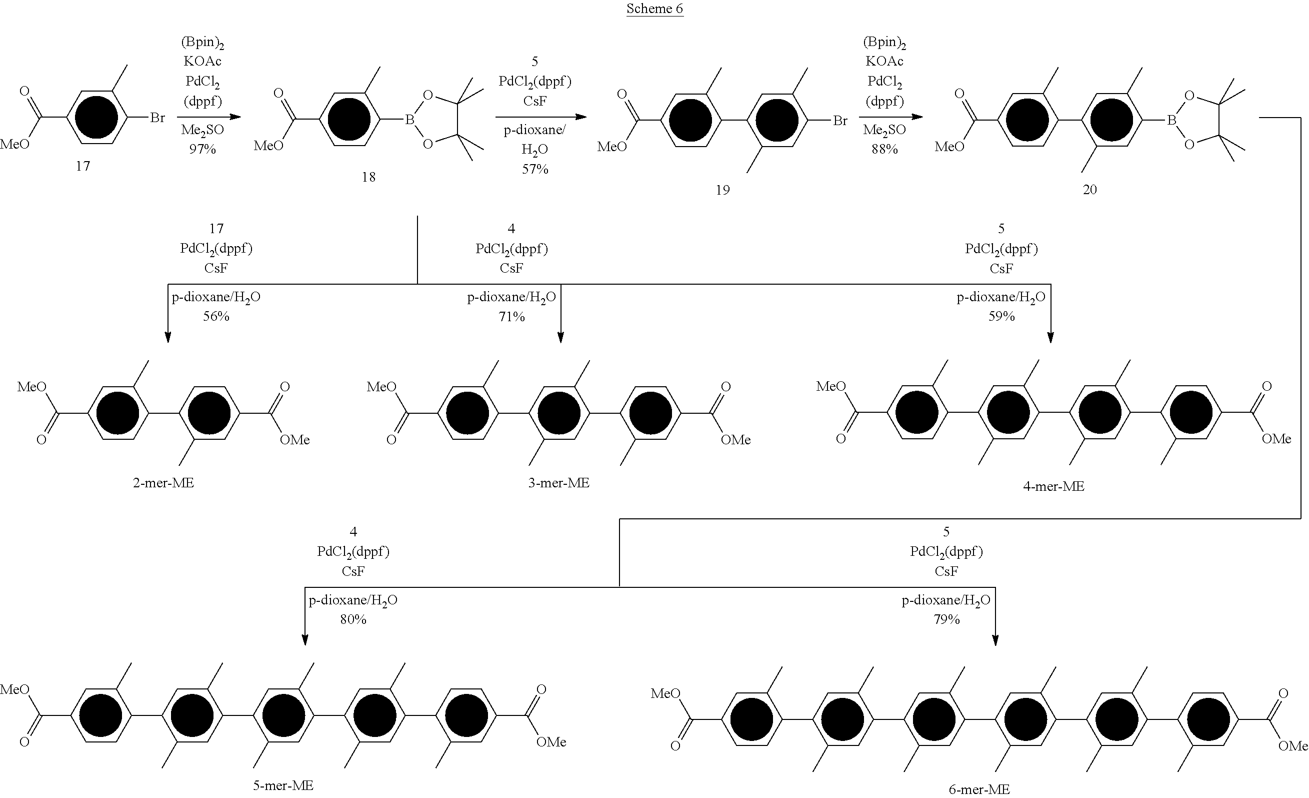

The reduced conjugation, induced by the twist between the planes of the neighboring phenylene rings is evident from spectrophotometric measurements. The absorption edge at around 300 nm in the UV/Vis absorbance spectra of compounds 2-mer-ME (n=2) to 6-mer-ME (n=6) do not change significantly on increasing the number of phenylene units from 2-6. Since a movement towards a lower energy of the absorption edge is expected[13h] on increasing the number of conjugated paraphenylenes, we conclude that the paraxylene units are somewhat isolated electronically. The fluorescence spectra on the other hand show a slight bathochromic shift as the oligomer length is increased from 2-4 rings. The emission max for the 4, 5, and 6 ring oligomers (2-mer-ME to 6-mer-ME), however, is only shifted ever so slightly, leading to the conclusion that the effective conjugation length in the excited state is reached at four rings. These findings agree with previously reported data.[13e]

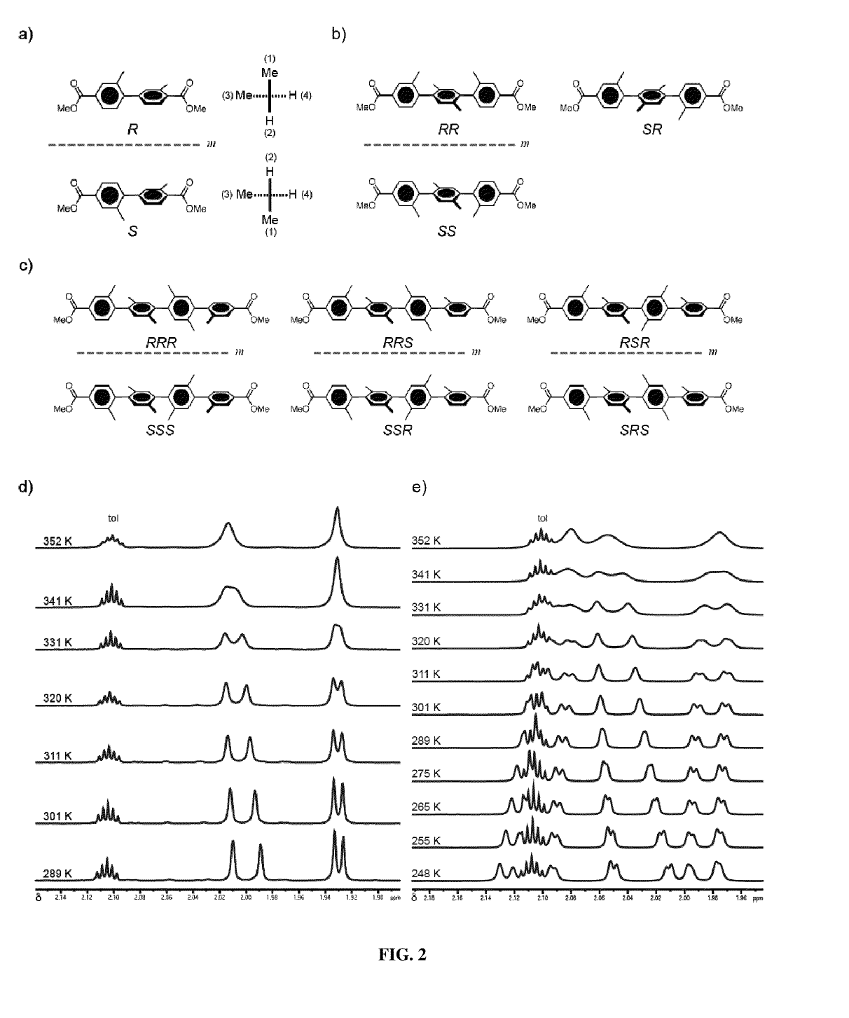

Since the steric hindrance between the ortho-methyl groups on each biphenyl subunit renders the planar conformation of the molecule an energy maximum and results in a twist between the planes of adjacent phenylene units, nonplanar isomers are generated with chiral axes whose helical sense is maintained as a result of hindered rotation about the single bonds. The simplest example with only two rotationally restricted phenylene units and one axis of chirality results in two enantiomers. When the number of contiguous rotationally restricted units is increased, however, a more complex mixture of atropisomers is obtained. In order to investigate and characterize these isomers, a series of model compounds (2-mer-ME-6-mer-Me) was prepared. They lack the hydroxyl group on the terminal phenylene rings, but have a methyl group in the meta position to the carbonyl group instead so as to hinder all the phenylene units rotationally. Since the rotational barrier is too low to isolate individual conformational isomers, 1H NMR spectroscopy was employed to gain a better understanding of the different conformations present in solution. We have used the methyl groups in the OPXs as 1H NMR probes of the stereochemistry. When 1H NMR spectroscopy was performed on the OPX oligomers 2-mer-ME to 6-mer-ME in order to characterize the isomers over a range of temperatures from 360 K down to 240 K, deuterated toluene (C₇D₈) was found to lead to the best resolution between signals for the probe methyl group protons in the different isomers generated by multiple axes of chirality in the oligomers from 3-mer-ME upwards. Thus, all 1H NMR spectra were recorded at 10 degree intervals in C₇D₈ after the samples had been left in the NMR probe to equilibrate at every temperature for 15 minutes. In the case of 2-mer-ME consisting of two paraxylene units and one chiral axis, two enantiomers are present, namely (R)-2-mer-ME and (S)-2mer-ME (FIG. 2a). As a consequence of the C2 symmetry present in both the (R)- and (S)-isomers, both methyl groups are homotopic by internal comparison and equivalent by external comparison and result in only one (isochronous) methyl resonance being observed in the 1H NMR spectrum of the racemic modification.

3-Mer-ME with two axes of chirality can exist (and does) as three isomers (FIG. 2b), namely (RR)-, (SS)- and (RS)-3-mer-ME in the ratio of 1:1:2. The two enantiomers (RR)- and (SS)-3-mer-ME have C2 symmetry and are, of course, chiral. The meso-isomer, (RS)-3-mer-ME has reflection symmetry (Ci) which means it is achiral. While the enantiomers behave as one compound in the ¹H NMR spectrum, they are diastereoisomeric with the meso-isomer. Thus, overall, in the case of the 3-mer-ME, two compounds can be identified in a mixture (and equilibrating) in C₇D₈ during VT ¹H NMR spectroscopy (FIG. 2d) at lower temperatures. Following some coalescence behavior at higher (˜380 K) temperatures, two broad resonances for the constitutionally heterotopic methyl group protons are observed at 352 K which both subsequently separate out again, giving a total of four equal intensity anisochronous signals for the two homotopic pairs in the enantiomers and the two enantiotopic pairs in the meso-isomer. A barrier to rotation of the paraxylene units linked to each other of about 18 kcal mol-1—consistent with previously reported values[13e, 15a]—was obtained using the Eyring equation[18]:

ΔG^{≠}=0.0191T_{c}(9.97+log(T_{c}/Δv)) (eqn 1)

Composed of four rotationally hindered paraxylene units, 4-mer-ME has three axes of chirality which lead to the existence (Table 1, FIG. 2c) of six different isomers—namely, three pairs of enantiomers, (RRR)-4-mer-ME/(SSS)-4-mer-ME, (RRS)-4-mer-ME/(SSR)-4-mer-ME, and (RSR)-4-mer-ME and (SRS)-4-mer-ME. The four conformational symmetrical (C2) isomers namely, the (RRR)-, (SSS)-, (RSR)- and (SRS)-isomers all contain three enantiotopic pairs of constitutionally heterotopic methyl groups giving rise to three anisochronous signals in the low temperature ¹H NMR spectrum for each enantiomeric pair, i.e., six resonances overall for the (RRR)-, (SSS)-, (RSR)- and (SRS)-isomers. In the case of the conformationally unsymmetrical (C1) enantiomers, (RRS)-4-mer-ME/(SSR)-4-mer-ME, all six methyl groups are heterotopic when atropisomerism is slow on the ¹H NMR time-scale and so give rise to six anisochronous resonances in total in the low temperature ¹H NMR spectrum. At high temperatures, three broad resonances are observed in keeping with the constitution of 4-mer-ME. On cooling down the solution, these three resonances first of all separate into six and then finally into 12 peaks (FIG. 2e). These 12 peaks constitute the sum of three peaks for the (RRR) and (SSS) enantiomers, three peaks for the (RSR) and (SRS) enantiomers and six peaks for the (RRS) and (SSR) enantiomers.

TABLE 1

| Compound | Isomer | Point Group | # Heterotopic Me^{[a]} |
|---|---|---|---|
| 2-mer-ME | R/S | C₂ | 1 |
| 3-mer-ME | RR/SS | C₂ | 4 |
| 3-mer-ME | RS | Cᵢ | \| |
| 4-mer-ME | RRR/SSS | C₂ | 12 |
| 4-mer-ME | RSR/SRS | C₂ | \| |
| 4-mer-ME | RRS/SSR | C₁ | \| |
| 5-mer-ME | RRRR/SSSS | C₂ | 32 |
| 5-mer-ME | RSSR/SRRS | C₂ | \| |
| 5-mer-ME | RRRS/SSSR | C₁ | \| |
| 5-mer-ME | RRSR/SSRS | C₁ | \| |
| 5-mer-ME | RRSS | Cᵢ | \| |
| 5-mer-ME | RSRS | Cᵢ | \| |
| 6-mer-ME | RRRRR/SSSSS | C₂ | 80 |
| 6-mer-ME | RRSRR/SSRSS | C₂ | \| |
| 6-mer-ME | RSSSR/SRRRS | C₂ | \| |
| 6-mer-ME | RSRSR/SRSRS | C₂ | \| |
| 6-mer-ME | RRRRS/SSSSR | C₁ | \| |
| 6-mer-ME | RRRSR/SSSRS | C₁ | \| |
| 6-mer-ME | RRRSS/SSSRR | C₁ | \| |
| 6-mer-ME | RRSSR/SSRRS | C₁ | \| |
| 6-mer-ME | RRSRS/SSRSR | C₁ | \| |
| 6-mer-ME | RSRRS/SRSSR | C₁ | \| |

^{[a]}# of heterotopic Me groups in 1H NMR spectroscopy for a mixture of all isomers of a certain length. The total number of heterotopic methyl peaks is displayed on the first entry of the respective n-mer-ME

The task of predicting the number of isomers (A) is not trivial since it depends on whether the number of chiral elements is odd or even. Losanitsch has already investigated the isomers in parafins and found the so-called Losanitsch series which can be described[19] in the context of the OPXs with the following equation; with A being the number of isomers, n the number phenylenes, and [x] the gauss bracket ([x]=largest integer not greater than x):

A=2⁽ⁿ⁻²⁾+2^{([n/2]-1)} (eqn 2)

While Losanitsch found the formula for the Losanitsch sequence to be eqn. 2 based on fitting empirical data, we have deduced an elegant way to describe eqn. 2 based on contemporary mathematical methods.

Increasing the number of phenylene units to n=5 and 6, the number of isomers are A=10 and 20, respectively. For 5-mer-ME (n=5), the 10 isomers lead to 32 anisochronous methyl groups, and for 6-mer-ME (n=6), the 20 isomers lead to 80 anisochronous methyl groups. It was not possible, however, to resolve all these methyl peaks in the VT-NMR spectra.

We have developed a highly efficient strategy for the synthesis of a series of oligoparaxylene rods terminated by carboxyl and hydroxyl groups and consisting of two up to eleven paraxylene rings linked in head-to-tail fashion to produce rigid, linear, partially conjugated ligands. The synthetic protocols adopted have relied heavily upon modular Suzuki-Miyaura cross-coupling reactions. Molecular gauge blocks based solely on paraxylene rings were found to be soluble in polar organic solvents all the way up to the homologue containing seven paraxylene rings. Ligands in which the central paraxylene rings are disubstituted with two hexyl groups instead of methyl groups were soluble up to the homologue containing nine benzenoid rings—throughout the odd series of three, five, seven and nine rings—all linked to each other in a head-to-tail manner. In the series of oligoparaxylene ligands with two hexyl groups on the central benzenoid ring, thermoreversible gelation was observed in dimethyl sulfoxide for the member of the series consisting of seven benzenoid rings. Finally, in order to achieve the syntheses of soluble ligands consisting of 11 benzenoid rings, two of the paraxylene rings—the fourth and eighth ones situated along the linear display—were substituted each with two hexyl groups instead of methyl groups. Theses constitutional features and selected modifications provide a suite of stable and rigid rods ranging from 5 all the way up to 50 Å in increments of roughly 5 Å.

One of the attractions of the oligoparaxylenes over the oligoparaphenylenes is their greatly increased solubilities because of the destabilization of the linear conjugated π-system framework in the former brought about by the mutual steric hindrance between near-neighbor methyl groups which force contiguous benzenoid rings out of planarity with each other. Another consequence of this twisting of the planes of the paraxylene rings is the introduction of axes of chirality between each of the benzenoid rings, leading to complex mixtures of atropisomers. A series of five model compounds in which the hydroxyl groups are no longer present on the terminal benzenoid rings, which instead carry methyl groups meta to methoxycarbonyl groups, were synthesized and investigated by dynamic ¹H NMR spectroscopy. These model 2-, 3-, 4-, 5- and 6-mers have, in turn, 2, 3, 6, 10 and 20 isomers with the possibilities of observing, in turn, resonances for 1, 4, 12, 32 and 80 anisochronous methyl groups. A partial unraveling of this complexity by low temperature ¹H NMR spectroscopy revealed that the barriers to rotation between the benzenoid rings are of the order of 18 kcal mol-1, i.e., not large enough to give rise to isolatable isomers at room temperature.

These rigid, linear, partially conjugated ligands can be used in a host of applications. So far, the oligoparaxylene gauge blocks have been introduced into isoreticular (IR)-MOF-74 extended structures. The six longest members of the series of IR-MOF-74 structures have the largest pore apertures of any metal-organic frameworks so far reported in the literature. It has been shown that large biomolecules such as vitamin B12, myoglobin and green fluorescent protein are able to pass through the pores of the larger IR-MOF-74 structures.

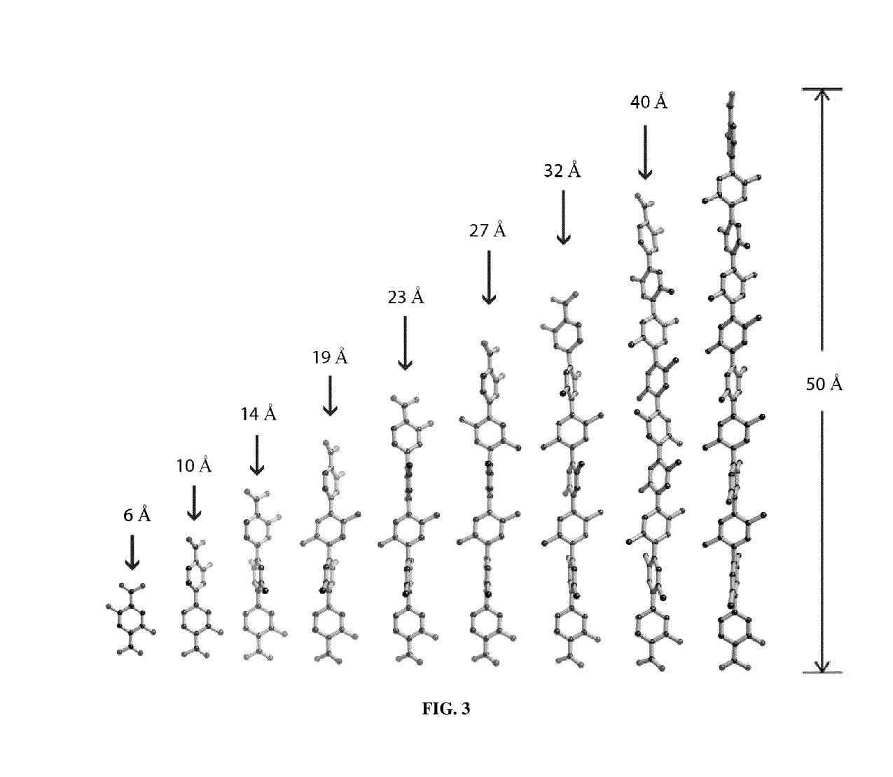

The limitations and scopes of preparing a molecular toolbox consisting of a large variety of building blocks has been explored, enabling the modular assembly of slender and robust organic rods with distinct and precise lengths, ranging from 0.5-5 nm (FIG. 3). The combination of the availability of several such building blocks and the possibility of assembling them in a modular fashion provides a rich toolbox of nanoscale gauge blocks. All of the reported gauge blocks have already been incorporated into metal-organic frameworks. This direct transmission of the shape onto the nano architectures and the wide modularity demonstrates that these molecular gauge blocks are very powerful tools for building on the nanoscale.

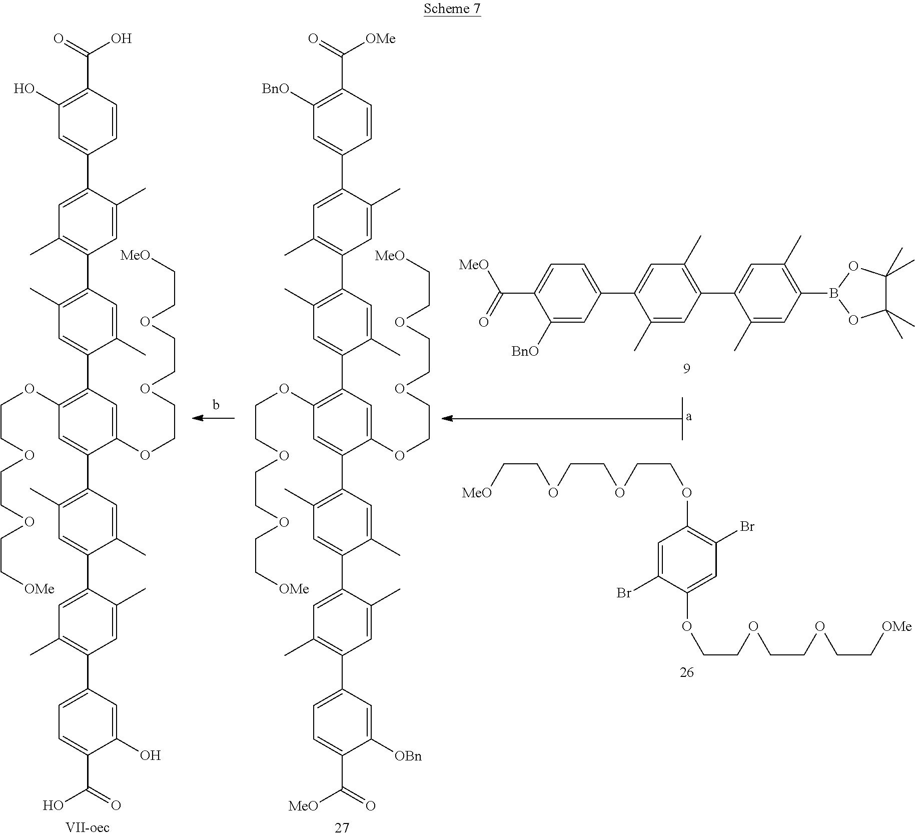 [SMILES: COCCOCCOCCOc1cc(-c2cc(C)c(-c3cc(C)c(-c4ccc(C(=O)O)c(O)c4)cc3C)cc2C)c(OCCOCCOCCOC)cc1-c1cc(C)c(-c2cc(C)c(-c3ccc(C(=O)O)c(O)c3)cc2C)cc1C ; COCCOCCOCCOc1cc(-c2cc(C)c(-c3cc(C)c(-c4ccc(C(=O)OC)c(OCc5ccccc5)c4)cc3C)cc2C)c(OCCOCCOCCOC)cc1-c1cc(C)c(-c2cc(C)c(-c3ccc(C(=O)OC)c(OCc4ccccc4)c3)cc2C)cc1C ; COC(=O)c1ccc(-c2cc(C)c(-c3cc(C)c(B4OC(C)(C)C(C)(C)O4)cc3C)cc2C)cc1OCc1ccccc1 ; COCCOCCOCCOc1cc(Br)c(OCCOCCOCCOC)cc1Br]

The triethylene glycol mono methyl ether substituted derivative VII-oeg was obtained by reacting the boronic ester building block 9 with the dibromide, which itself was observed in two steps starting from commercially available 2,5-dibromohydroquinone following a reported procedure. The benzylic ether protection groups were then cleaved by hydrogenolysis using Raney Ni as a catalyst. Subsequently the methyl ester groups were saponified to yield the target compound VII-oegas a colorless solid. The synthesis the ethylene glycol-substituted OPX link VII-oeg is summarized in Scheme 7: Reagents and conditions: a) CsF, PdCl₂(dppf), p-dioxane/H₂O, 100° C. b) i) Raney N₁, H₂, THF, 50° C.; ii) NaOH, THF/H₂O, 50° C.

### EXAMPLES

Materials and Methods: Anhydrous tetrahydrofuran (THF), dichloromethane (CH₂Cl₂) and acetonitrile (MeCN) were obtained from an EMD Chemicals DrySolv® system. Anhydrous p-dioxane and Me2SO were purchased from Aldrich and stored under an atmosphere of argon. CDCl3, C6D6, CD2Cl2, THF-d8 and (CD3)2CO were purchased from Aldrich and used without further purification. All other reagents and solvents were purchased from commercial sources and were used without further purification, unless indicated otherwise. All reactions were carried out under an atmosphere of N₂ in flame-dried flasks using anhydrous solvents, unless indicated otherwise. Thin-layer chromatography (TLC) was carried out using glass or aluminum plates, precoated with silica-gel 60 containing fluorescent indicator (Whatman LK6F). The plates were inspected by UV light (254 nm) and/or KMnO4 stain. Column chromatography was carried out employing the flash technique using silica-gel 60F (230-400 mesh). ¹H and ¹³C NMR spectra were recorded on a Bruker ARX500 (500 MHz) spectrometer. UV/Vis spectra were recorded on a Shimadzu UV-3600 spectrophotometer. Fluorescence spectra were recorded on a Shimadzu RF-5301PC spectrofluorometer. VT-NMR spectra were recorder on a Bruker Avance 600 MHz spectrometer, which was temperature-calibrated using neat ethylene glycol or MeOH. The chemical shifts (δ) for ¹H spectra, given in ppm, are referenced to the residual proton signal of the deuterated solvent. The chemical shifts (δ) for ¹³C spectra are referenced relative to the signal from the carbon of the deuterated solvent. SEM imaging was performed on a FEI Quanta 600F sFEG ESEM scanning electron microscope. High-resolution mass spectra were measured on a Finnigan LCQ iontrap mass spectrometer (HR-ESI).

### General Synthetic Procedures

Suzuki-Miyaura Cross-Couplings: A p-dioxane/H₂O mixture (2:1 v/v, 0.12 M based on the aryl halide) was purged with N2 and transferred subsequently via a cannula to a round-bottomed flask charged with the aryl halide (1.00 equiv), the boronic acid pinacol ester (1.10 equiv per halide), CsF (3.00 equiv per halide) and (dppf)PdCl₂ (5 mol % per halide). The resulting mixture was heated under reflux overnight. It was then cooled to rt and the products were purified using techniques outlined with specific measures for the individual reactions discussed in the section on synthetic procedures.

Hydrogenolysis: The starting material was dissolved in anhydrous THF. The Pd/C catalyst was added (10 w %) under an atmosphere of N₂. The reaction mixture was stirred overnight under an atmosphere of H₂, filtered over a Celite plug, washed with solvent and concentrated to give the crude product which was used directly in the next step.

Saponifications: The starting material was stirred in a THF/aq. 0.5 M NaOH (1:1 v/v) mixture at 50° C. overnight. The THF was then removed in vacuo to provide typically an insoluble white solid in H₂O. While stirring, the aqueous layer was acidified with concentrated HCl until a pH<2 was attained and the resulting precipitate was collected by vacuum filtration, washed with ample H₂O and air-dried for 24 h to provide the target compound as a white powder.

### Synthetic Procedures

Methyl 2-(Benzyloxy)-4-iodobenzoate (2): Solid K₂CO₃ (76.1 g, 0.55 mol) was added to a solution of methyl 4-iodosalicylate (1) (76.5 g, 0.28 mol) in MeCN (550 mL) at rt. Benzyl bromide (32.7 mL, 0.28 mol) was added via a syringe, and the resulting reaction mixture was warmed to 80° C. and stirred overnight. The reaction mixture was then cooled to rt and filtered subsequently to remove insoluble salts, which were washed further with EtOAc. The filtrate was concentrated in vacuo, before redissolving the residue in fresh EtOAc and filtering it a second time. The filtrate was concentrated in vacuo to provide an oil which solidified upon standing to yield 2 (100.5 g, 99%) as a beige solid which required no further purification. ¹H NMR (500 MHz, CDCl₃, 25° C.) δ=7.54 (d, J=8.0 Hz, 1H), 7.49 (d, J=7.5 Hz, 2H), 7.43-7.35 (m, 4H), 7.33 (t, J=7.5 Hz, 1H), 5.15 (s, 2H), 3.89 (s, 3H) ppm. ¹³C NMR (126 MHz, CDCl₃, 25° C.) δ=166.3, 158.4, 136.2, 133.1, 130.1, 128.8, 128.1, 127.0, 123.3, 120.3, 100.0, 71.0, 52.3 ppm. HRMS (ESI) Calcd for C₁₅H₁₄IO₃: m/z=367.9909 ([M+H]+); Found m/z=367.9893.

3: Anhydrous p-dioxane (1.35 L) was added to a three-neck round-bottomed flask equipped with a reflux condenser and charged with methyl 2-(benzyloxy)-4-iodobenzoate (100.2 g, 0.27 mol), bis(pinacolato)diboron (76.0 g, 0.30 mol), KOAc (80.1 g, 0.82 mol) and (Ph₃P)₂PdCl₂ (3.8 g, 5.4 mmol). The reaction mixture was purged at rt with dry N₂ (approx. 30 min) and the flask was transferred to an oil-bath pre-warmed to 130° C. The reaction mixture was heated under reflux for 16 h, before being cooled to rt and filtered to remove insoluble salts which were washed further with EtOAc. The filtrate was concentrated in vacuo and the residue was dissolved in EtOAc. Ample activated carbon was added to the dark brown/black solution and the EtOAc solution was warmed to reflux for 15 min. The insoluble material was removed by hot filtration through a pad of Celite to provide a yellow solution which was concentrated in vacuo to provide an oil. To facilitate solidification, hexanes (200 mL) were added and the solution was concentrated in vacuo. To remove any residual p-dioxane and/or EtOAc, this coevaporation process with hexanes was repeated a further two times, to yield a light brown solid which is insoluble in hexanes at rt. Hexanes (500 mL) was added to the solid and warmed to reflux to provide a homogeneous solution that was removed from the heat source and left to stand overnight to crystallize. Filtration provided 3 (78.1 g, 78%) as yellow needles. ¹H NMR (500 MHz, CDCl₃, 25° C.) δ=7.80 (d, J=7.5 Hz, 1H), 7.53 (d, J=7.5 Hz, 2H), 7.48 (s, 1H), 7.44 (dd, J=7.5, 1.0 Hz, 1H), 7.40 (t, J=8.0 Hz, 2H), 7.31 (t, J=7.5 Hz, 1H), 5.22 (s, 2H), 3.90 (s, 3H), 1.36 (s, 12H) ppm. ¹³C NMR (126 MHz, CDCl₃, 25° C.) δ=166.9, 157.4, 136.9, 134.7 (br.), 130.9, 128.5, 127.7, 127.0, 126.9, 123.1, 119.3, 84.3, 70.6, 52.1, 24.9 ppm. HRMS (ESI) Calcd for C₂₁H₂₅BlO₅: m/z=369.1873 ([M+H]+), Found m/z=369.1884; m/z=391.1693 ([M+Na]+); Found m/z=369.1702.

2-Mer-BME: By following the General Coupling Procedure (based on 4.00 g of the aryl iodide), the desired product precipitated out of solution. The aqueous (bottom) layer of the biphasic solution was removed via a syringe, and the product was collected by filtration of the remaining dark brown dioxane layer. The collected product was washed with EtOAc and allowed to air dry to provide the 2-mer-BME (4.35 g, 83%) as an off-white powder which required no further purification. ¹H NMR (500 MHz, CDCl₃, 25° C.) δ=7.91 (d, J=8.0 Hz, 2H), 7.52 (d, J=7.5 Hz, 4H), 7.42 (t, J=7.5 Hz, 4H), 7.34 (t, J=7.5 Hz, 2H), 7.16 (dd, J=8.0, 1.5 Hz, 2H), 7.08 (d, J=1.5 Hz, 2H), 5.23 (s, 4H), 3.93 (s, 6H) ppm. ¹³C NMR (125 MHz, CDCl₃, 25° C.) δ=166.5, 158.6, 145.4, 136.7, 132.6, 128.8, 128.1, 127.0, 120.2, 119.6, 113.1, 70.9, 52.3 ppm. MS (ESI) Calcd for C₃₀H₁₆O₆: m/z=483.2 ([M+H]+), 505.2 ([M+Na]+) and 987.3 ([2M+Na]+; Found m/z=483.4 ([M+H]+), 505.4 ([M+Na]+) and 987.1 ([2M+Na]+).

2-Mer-HCA: EtOAc (50 mL), THF (50 mL) was used as a solvent mixture. Pd/C (0.98 g, 0.90 mmol) was added to a flask charged with the 2-mer-BME (4.35 g, 9.0 mmol) and the General Hydrogenolysis Conditions were followed. The filter cake was suspended in THF (30 mL) and the suspension was warmed to reflux and hot-filtered subsequently. This process was repeated five times in order to extract all the product from the Pd/C. The combined organic layers were concentrated in vacuo to provide the product which was used directly in the next step. Following the General Saponification Procedure, 2.0 g of the 2-mer-HCA (82% over hydrogenolysis and saponification) were collected as a white solid. ¹H NMR (500 MHz, CD₃SOCD₃, 25° C.) δ=7.86 (d, J=9.0 Hz, 2H), 7.30-7.25 (m, 4H) ppm. ¹³C NMR (126 MHz, CD₃SOCD₃, 25° C.) δ=171.7, 161.4, 145.5, 131.0, 117.9, 115.3, 113.0 ppm. HRMS (ESI) Calcd for C₁₄H₁₀O₆: m/z=273.0405 ([M−H]−); Found m/z=273.0397.

3-Mer-BME: Following the General Coupling Procedure (based on 2.00 g of the aryl dibromide), the desired product precipitated out of solution. The aqueous (bottom) layer of the biphasic solution was removed via a syringe, and the product was collected by filtration of the remaining dark brown dioxane layer. The collected product was washed with EtOAc and left to dry in air to provide the 3-mer-BME (4.29 g, 96%) as an off-white powder which required no further purification. ¹H NMR (500 MHz, THF-d₈, 25° C.) δ=7.96 (d, J=7.5 Hz, 2H), 7.66 (d, J=7.5 Hz, 4H), 7.48 (t, J=7.5 Hz, 4H), 7.39 (t, J=7.5 Hz, 2H), 7.24 (s, 2H), 7.22 (s, 2H), 7.10 (dd, J=8.0, 1.0 Hz, 2H), 5.36 (s, 4H), 3.98 (s, 6H), 2.30 (s, 6H) ppm. ¹³C NMR (125 MHz, THF-d₈, 25° C.) δ=167.0, 159.3, 148.2, 141.8, 138.8, 133.7, 132.6, 132.6, 129.5, 128.6, 127.9, 122.2, 120.8, 116.1, 71.4, 52.2, 20.3 ppm. MS (ESI) Calcd for C₃₈H₃₄O₆: m/z=587.2 ([M+H]+), 609.2 ([M+Na]+) and 1195.5 ([2M+Na]+; Found m/z=587.4 ([M+H]+), 609.5 ([M+Na]+) and 1195.1 ([2M+Na]+).

3-Mer-HCA: Following the General Hydrogenolysis Conditions but using a DMF (15 mL)/THF (60 mL) mixture, Pd/C (0.78 g, 0.73 mmol) was added to a flask charged with 3-mer-BME (4.29 g, 7.32 mmol). The filter cake was washed with THF (10 mL) and the combined filtrate was concentrated in vacuo to provide the product that was used directly in the next step. Following the General Saponification Procedure, the 3-mer-HCA (2.4 g, 87% over hydrogenolysis and saponification) was isolated as a white powder. 1H NMR (500 MHz, CD3SOCD3, 25° C.) δ=14.0 (br. s, 2H), 11.4 (br. s, 2H), 7.85-7.82 (m, 2H), 7.18 (s, 2H), 6.98-6.92 (m, 4H), 2.24 (m, 6H) ppm. 13C NMR (126 MHz, CD3SOCD3, 25° C.) δ=171.9, 160.9, 148.3, 139.7, 132.3, 131.3, 130.2, 120.3, 117.4, 111.7, 19.5 ppm. HRMS (ESI) Calcd for C22H17O6: m/z=377.1031 ([M−H]−); Found m/z=377.1031.

4-Mer-BME: By following the General Coupling Procedure, 3 (4.78 g, 13.0 mmol, 3.0 equiv) and 5 (2.00 g, 4.33 mmol, 1.0 equiv) were reacted in a degassed 2:1 dioxane/H2O mixture (420 mL) with CsF (3.95 g, 26.0 mmol, 6.0 equiv) and PdCl2(dppf) (354 mg, 0.433 mmol, 10 mol %). The reaction mixture was cooled to rt, before being extracted with CH2Cl2 and H2O. The aqueous phase was washed twice with CH2Cl2. The combined organic phases were dried (MgSO4), filtered and concentrated to afford the crude product which was absorbed on silica-gel and subjected to column chromatography (SiO2: hexanes:EtOAc=5:1) to give the 4-mer-BME as a colorless solid (2.60 g, 3.82 mmol, 87%). 1H NMR (500 MHz, CD2Cl2, 25° C.): δ=7.86 (d, J=7.9 Hz, 2H), 7.51 (m, 4H), 7.40 (m, 4H), 7.33 (m, 2H), 7.13 (m, 2H), 7.06 (d, J=1.3 Hz, 2H), 7.03 (dd, J=7.9, 1.5 Hz, 2H), 7.01 (s, 2H), 5.29 (s, 4H), 3.89 (s, 6H), 2.20 (s, 6H), 2.09 (s, 6H) ppm. 13C NMR (126 MHz, CD2Cl2, 25° C.): δ=166.8, 158.2, 147.7, 141.2, 140.0, 137.3, 133.7, 132.6, 131.9, 131.8, 131.2, 128.9, 128.2, 127.4, 121.9, 119.5, 115.4, 71.0, 52.3, 20.0, 19.5 ppm. HRMS (ESI): m/z calcd for C46H43O6 [M+H]+ 691.3051; found 691.3067.

4-Mer-HCA: Following the General Hydrogenolysis Procedure but using an EtOAc (10 mL)/EtOH (10 mL) mixture, Pd/C (0.15 g, 0.15 mmol) was added to a flask charged with the 4-mer-BME (1.00 g, 1.45 mmol). The filter cake was washed with THF (10 mL) and the combined filtrate was concentrated in vacuo to provide the product that was used directly in the next step. Following the General Saponification Procedure, 620 mg of the 4-mer-HCA (89% over hydrogenolysis and saponification) was collected as a white solid. 1H NMR (500 MHz, CD3SOCD3, 25° C.) δ=7.86-7.83 (m, 2H), 7.19 (s, 2H), 7.06 (s, 2H), 6.98-6.92 (m, 4H), 2.25 (s, 6H), 2.06 (s, 6H) ppm. 13C NMR (126 MHz, CD3SOCD3) δ=171.8, 160.9, 148.5, 140.3, 138.9, 132.8, 131.8, 131.3, 130.6, 130.1, 120.3, 117.3, 111.5, 19.6, 19.0 ppm. HRMS (ESI) Calcd for C30H26O6: m/z=481.1657 ([M−H]−); Found m/z=481.1654.

6: Following the General Coupling Procedure, 3 (9.20 g, 25.0 mmol, 1.0 equiv) and 4 (35.9 g, 135.8 mmol, 5.4 equiv) were reacted in a degassed 2:1 dioxane/H2O mixture (300 mL) with CsF (11.4 g, 75.0 mmol, 3.0 equiv) and PdCl2(dppf) (1.11 g, 1.36 mmol, 5 mol %). The reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:CH2Cl2=1:1-1:10) to give the compound 6 as a yellowish oil (7.75 g, 18.2 mmol, 67%). 1H NMR (500 MHz, CDCl3, 25° C.): δ=7.87 (d, J=7.9 Hz, 1H), 7.48 (m, 2H), 7.43 (s, 1H), 7.40 (m, 2H), 7.31 (m, 1H), 7.03 (s, 1H), 6.91 (dd, J=7.9, 1.5 Hz, 1H), 6.89 (d, J=1.3 Hz, 1H), 5.21 (s, 2H), 3.93 (s, 3H), 2.38 (s, 3H), 2.08 (s, 3H) ppm. 13C NMR (126 MHz, CDCl3, 25° C.): δ=166.7, 157.8, 146.4, 140.0, 136.6, 135.2, 134.5, 133.9, 131.8, 131.5, 128.6, 127.8, 126.8, 124.2, 121.3, 119.2, 114.9, 70.6, 52.1, 22.3, 19.5 ppm. HRMS (ESI): m/z calcd for C23H22BrO3 [M+H]+ 425.0747; found 425.0755.

7: 6 (7.75 g, 18.2 mmol, 1.0 equiv) was dissolved in dry and degassed Me2SO (80 mL). Bis(pinacolata)diboron (5.08 g, 20.0 mmol, 1.1 equiv), KOAc (5.36 g, 54.6 mmol, 3.0 equiv) and PdCl2(dppf) (743 mg, 0.91 mmol, 5 mol %) were added and the reaction mixture was heated to 80° C. for 14 h before being cooled to rt and extracted with CH2Cl2 and H2O. The aqueous phase was washed twice with CH2Cl2. The combined organic phases were washed with H2O, dried (MgSO4), filtered and concentrated. The crude product was absorbed on silica-gel and subjected to column chromatography (SiO2: hexanes:EtOAc=5:1) to afford compound 7 as a colorless oil (8.04 g, 17.0 mmol, 94%). 1H NMR (500 MHz, CDCl3, 25° C.): δ=7.91 (d, J=7.8 Hz, 1H), 7.68 (s, 1H), 7.51 (m, 2H), 7.41 (m, 2H), 7.33 (m, 1H), 7.02 (s, 1H), 6.98-6.95 (m, 2H), 5.23 (s, 2H), 3.96 (s, 3H), 2.55 (s, 3H), 2.14 (s, 3H), 1.39 (s, 12H) ppm. 13C NMR (126 MHz, CDCl3, 25° C.): =166.7, 157.8, 147.4, 143.1, 142.4, 138.1, 136.7, 131.7, 131.3, 130.8, 128.6, 127.8, 126.8, 121.3, 119.0, 114.9, 83.5, 70.6, 52.1, 25.1, 24.9, 21.7, 19.5 ppm. HRMS (ESI): m/z calcd for C29H34BO6 [M+H]+ 472.2544; found 472.2530.

8: Following the General Coupling Procedure, 3 (1.08 g, 2.92 mmol, 1.0 equiv) and 5 (5.40 g, 11.7 mmol, 4.0 equiv) were reacted in degassed dioxane (150 mL) and H2O (50 mL) with CsF (1.33 g, 8.76 mmol, 3.0 equiv) and PdCl2(dppf) (120 mg, 0.147 mmol, 5 mol %). The reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=5:1) to give the compound 8 as a colorless solid (1.11 g, 1.93 mmol, 66%). 1H NMR (500 MHz, CDCl3, 25° C.): δ=7.90 (d, J=8.2 Hz, 1H), 7.74 (s, 1H), 7.51 (d, J=8.5 Hz, 2H), 7.40 (t, J=7.6 Hz, 2H), 7.32 (t, J=7.3 Hz, 1H), 7.07 (s, 1H), 7.02-7.00 (m, 3H), 6.95 (s, 1H), 5.24 (s, 2H, CH2), 3.94 (s, 3H, CH3), 2.41 (s, 3H), 2.15 (s, 3H), 2.04 (m, 6H) ppm. 13C NMR (126 MHz, CDCl3, 25° C.): δ=166.8, 157.8, 147.3, 141.4, 140.2, 139.88, 139.81, 138.5, 136.8, 135.4, 133.2, 132.3, 131.7, 131.3, 130.9, 130.5, 128.6, 127.8, 126.8, 121.6, 118.9, 115.0, 99.6, 70.6, 52.1, 27.5, 19.8, 19.3, 19.0 ppm. HRMS (ESI): m/z calcd for C31H30IO3 [M+H]+ 577.1234; found 577.1229.

9: 8 (971 mg, 1.68 mmol, 1.0 equiv) was dissolved in dry and degassed Me2SO (7 mL). Bis(pinacolata)diboron (470 mg, 1.85 mmol, 1.1 equiv), KOAc (494 mg, 5.04 mmol, 3.0 equiv) and PdCl2(PPh3)2 (69 mg, 0.084 mmol, 5 mol %) were added and the reaction mixture was heated to 80° C. for 15 h. After cooling to rt, it was extracted with H2O and CH2Cl2. The aqueous phase was washed twice with CH2Cl2. The combined organic phases were washed with H2O, dried (MgSO4), evaporated and subjected to column chromatography (SiO2: hexanes:EtOAc=9:1), to give compound 9 as a colorless foam (810 mg, 1.41 mmol, 84%). 1H NMR (500 MHz, CDCl3, 25° C.): δ=7.93 (d, J=8.2 Hz, 1H), 7.70 (s, 1H), 7.54 (d, J=8.0 Hz, 2H), 7.42 (t, J=7.5 Hz, 2H), 7.35 (t, J=7.3 Hz, 1H), 7.10 (s, 1H), 7.05 (m, 2H), 6.99 (s, 1H), 6.98 (s, 1H), 5.27 (s, 2H), 3.97 (s, 3H), 2.55 (s, 3H), 2.17 (s, 3H), 2.10 (s, 3H), 2.06 (s, 3H), 1.39 (s, 12H) ppm. 13C NMR (126 MHz, CDCl3, 25° C.): δ=166.8, 157.8, 147.5, 143.8, 142.0, 141.3, 139.5, 137.4, 136.8, 133.2, 132.1, 132.0, 131.7, 131.2, 130.9, 130.7, 128.6, 127.8, 126.8, 121.7, 118.8, 115.1, 83.5, 70.6, 52.1, 34.7, 31.6, 25.3, 24.98, 24.95, 22.7, 21.8, 20.8, 19.8, 19.3, 19.2, 14.3 ppm. HRMS (ESI): m/z calcd for C37H41BO5 [M+H]+ 576.3156; found 576.3169.

10: Following the General Coupling Procedure, 7 (2.60 g, 5.50 mmol, 1.0 equiv) and 5 (10.2 g, 22.1 mmol, 4.0 equiv) were dissolved in a degassed mixture of dioxane (500 mL) and H2O (200 mL) at 80° C. before CsF (2.50 g, 16.5 mmol, 3.0 equiv) and PdCl2(dppf) (225 mg, 0.276 mmol, 5 mol %) were added. After the reaction was complete, the mixture was cooled to rt before being extracted with CH2Cl2 (400 mL) and H2O (1000 mL). The aqueous phase was washed twice with CH2Cl2 (2×200 mL). The combined organic phases were washed with brine, dried (MgSO4) and evaporated. The crude product was subjected to column chromatography (SiO2: hexanes:EtOAc=5:1) to give the product as a colorless oil (2.34 g, 3.44 mmol, 63%). 1H NMR (500 MHz, CDCl3, 25° C.): δ=7.94 (d, J=8.2 Hz, 1H), 7.77 (s, 1H), 7.55 (d, J=7.4 Hz, 2H), 7.43 (t, J=7.6 Hz, 2H), 7.35 (t, J=7.3 Hz, 1H), 7.13 (s, 1H), 7.09-7.03 (m, 5H), 6.99 (m, 1H), 5.27 (s, 2H), 3.97 (s, 3H), 2.45 (s, 3H), 2.20 (s, 3H), 2.14-2.06 (m, 12H) ppm. 13C NMR (126 MHz, CDCl3, 25° C.): δ=166.8, 157.9, 147.5, 141.83, 141.77, 141.20, 141.14, 141.00, 140.1, 139.8, 139.53, 139.50, 139.41, 139.36, 138.39, 138.36, 136.8, 135.56, 135.49, 133.45, 133.39, 132.92, 132.83, 132.66, 132.56, 132.1, 131.66, 131.60, 131.54, 130.78, 130.73, 130.69, 130.62, 130.48, 130.45, 128.6, 127.8, 126.8, 121.7, 118.8, 115.1, 99.4, 70.6, 52.1, 34.70, 34.57, 27.4, 25.3, 20.7, 19.83, 19.79, 19.47, 19.44, 19.37, 19.31, 18.98, 18.85 ppm. HRMS (ESI): m/z calcd for C39H38IO3 [M+H]+ 681.1860; found 681.1848.

11: 10 (2.06 g, 3.02 mmol, 1.0 equiv) was dissolved in dry and degassed Me2SO (20 mL). Bis(pinacolato)diboron (843 mg, 3.32 mmol, 1.1 equiv), KOAc (888 mg, 9.06 mmol, 3.0 equiv) and PdCl2(dppf) (123 mg, 0.151 mmol, 5 mol %) were added and the reaction mixture was heated to 80° C. for 14 h before being cooled to rt and extracted with CH2Cl2 and H2O. The aqueous phase was washed twice with CH2Cl2. The combined organic phases were washed with H2O, dried (MgSO4), filtered and concentrated. The crude product was absorbed on silica-gel and subjected to column chromatography (SiO2: hexanes:EtOAc=7:1) to give compound 11 as a colorless solid (1.50 g, 2.2 mmol, 72%). 1H NMR (500 MHz, CDCl3, 25° C.): δ=7.94 (d, J=8.3 Hz, 1H), 7.71 (s, 1H), 7.55 (d, J=7.7 Hz, 2H), 7.44-7.41 (m, 2H), 7.35 (m, 1H), 7.12 (s, 1H), 7.10-6.99 (m, 6H), 5.27 (s, 2H), 3.97 (s, 3H), 2.57 (s, 3H), 2.20 (s, 3H), 2.14-2.06 (m, 12H), 1.39 (s, 12H) ppm. 13C NMR (126 MHz, CDCl3, 25° C.): δ=166.8, 157.9, 147.6, 144.25, 144.19, 141.95, 141.93, 141.38, 141.31, 140.49, 140.44, 139.87, 139.82, 139.44, 139.41, 137.3, 136.8, 133.52, 133.46, 132.72, 132.65, 132.62, 132.55, 132.19, 132.14, 132.06, 132.04, 131.66, 131.60, 131.04, 130.96, 130.75, 130.61, 130.59, 130.40, 130.38, 128.6, 127.8, 126.8, 121.7, 118.8, 115.11, 115.10, 83.4, 77.1, 70.6, 52.1, 31.6, 25.06, 24.98, 24.96, 21.76, 21.73, 19.82, 19.78, 19.49, 19.45, 19.36, 19.31, 19.21, 19.08 ppm. HRMS (ESI): m/z calcd for C45HSOB05 [M+H]+ 680.3782; found 680.3776.

5-Mer-BME: Following the General Coupling Procedure, 7 (1.77 g, 3.75 mmol, 3.0 equiv) and 1,4-diiodo-2,5-dimethyl benzene[20] (447 mg, 1.25 mmol, 1.0 equiv) were dissolved in a degassed 2:1 dioxane/H2O mixture (150 mL) and reacted with CsF (1.14 g, 7.50 mmol, 6.0 equiv) and PdCl2(dppf) (102 mg, 0.125 mmol, 10 mol %). After completion of the reaction, the reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=5:1) to give the 5-mer-BME as a colorless solid (765 mg, 0.962 mmol, 77%). 1H NMR (500 MHz, CDCl3, 25° C.): δ=7.92 (d, J=8.2 Hz, 2H), 7.53 (m, 4H), 7.41 (m, 4H), 7.32 (m, 2H), 7.11 (s, 2H), 7.09 (m, 2H), 7.05-7.03 (m, 6H), 5.25 (s, 4H), 3.95 (s, 6H), 2.19 (s, 6H), 2.13 (s, 3H), 2.12 (s, 3H), 2.11 (m, 6H) ppm. 13C NMR (126 MHz, CDCl3, 25° C.): δ=166.8, 157.9, 147.6, 141.30, 141.23, 140.03, 139.98, 139.50, 139.47, 136.8, 133.50, 133.44, 132.84, 132.74, 132.10, 132.08, 131.67, 131.60, 130.79, 130.72, 130.70, 128.6, 127.8, 126.8, 121.7, 118.8, 115.11, 115.10, 70.6, 52.1, 19.84, 19.80, 19.51, 19.47, 19.34 ppm. HRMS (ESI): m/z calcd for C54H51O6 [M+H]+ 795.3680; found 795.3659.

5-Mer-HCA: Following the General Hydrogenolysis Procedure with THF (20 mL), Pd/C (0.29 g, 0.27 mmol) and 5-mer-BME (2.16 g, 2.72 mmol), the product was obtained as a colorless solid and used directly in the next step. Following the General Saponification Procedure, 1.60 g of the 5-mer-HCA (quantitative over hydrogenolysis and saponification) was collected as an off-white solid. 1H NMR (500 MHz, DMF-d7, 25° C.) δ=8.00 (d, J=8.0 Hz, 2H), 7.31 (s, 2H), 7.16-7.12 (m, 4H), 7.07 (dd, J=8.0, 1.5 Hz, 2H), 7.04-7.02 (m, 2H), 2.37 (s, 6H), 2.18-2.16 (m, 6H), 2.15 (s, 6H). 13C NMR (126 MHz, DMF-d7, 25° C.) δ=172.6, 149.2, 141.2, 140.20, 140.18, 139.5, 133.38, 133.35, 132.8, 132.2, 131.6, 130.90, 130.88, 130.78, 130.75, 130.3, 120.5, 117.6, 111.9, 19.38, 19.36, 18.82, 18.79, 18.7 ppm. 1H NMR (600 MHz; CD3SOCD3, 100° C.): δ=7.86 (d, J=8.6 Hz, 2H), 7.16 (s, 2H), 7.07 (s, 2H), 7.04 (s, 2H), 6.94-6.93 (m, 4H), 2.26 (s, 6H), 2.08 (s, 6H), 2.07 (s, 6H) ppm. 13C NMR (151 MHz; CD3SOCD3, 100° C.): δ=171.8, 161.3, 149.2, 141.2, 140.2, 139.6, 133.3, 132.8, 132.1, 131.8, 130.98, 130.93, 130.6, 120.7, 117.9, 112.6, 19.8, 19.39, 19.27 ppm. HRMS (ESI) Calcd for C38H34O6: m/z=585.2283 ([M−H]−); Found m/z=585.2299.

6-Mer-BME: Following the General Coupling Procedure, 7 (600 mg, 1.27 mmol, 3.0 equiv) and 5 (196 mg, 0.423 mmol, 1.0 equiv) were dissolved in a degassed 2:1 dioxane/H2O mixture (45 mL) and reacted with CsF (386 mg, 2.54 mmol, 6.0 equiv) and PdCl2(dppf) (35 mg, 0.043 mmol, 10 mol %). After completion of the reaction, the mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=5:1) to give the 6-mer-BME as a colorless solid (229 mg, 0.255 mmol, 60%). 1H NMR (500 MHz, CDCl3, 25° C.): δ=7.93 (d, J=8.3 Hz, 2H), 7.53 (m, 4H), 7.40 (m, 4H), 7.33 (m, 2H), 7.12-7.04 (m, 12H), 5.26 (s, 4H), 3.95 (s, 6H), 2.19 (s, 6H), 2.15-2.12 (m, 18H) ppm. 13C NMR (126 MHz, CDCl3, 25° C.): δ=166.8, 157.9, 147.6, 141.4, 141.3, 140.8, 140.5, 140.44, 140.43, 140.38, 139.9, 139.83, 139.82, 139.78, 139.7, 139.5, 139.4, 136.81, 136.78, 133.54, 133.48, 133.9, 132.93, 132.88, 132.82, 132.75, 132.72, 132.65, 132.6, 132.08, 132.06, 131.71, 131.67, 131.5, 130.9, 130.83, 130.78, 130.7, 128.6, 127.8, 126.8, 121.7, 118.8, 115.1, 70.6, 52.1, 19.9, 19.8, 19.53, 1949, 19.47, 19.46, 19.38, 19.36 ppm. HRMS (ESI): m/z calcd for C62H59O6 [M+H]+ 899.4306; found 899.4330.

6-mer-HCA: Following the General Hydrogenolysis Procedure in THF (60 mL) with Raney Ni as catalyst (approx. 200 mg, commercially purchased as a slurry in H2O which was converted to a slurry in THF by successive (5×) dilutions with THF, followed by removal of the supernatant) and the 6-mer-BME (2.0 g, 2.22 mmol). The reaction mixture was stirred for 48 h before being cooled to rt and purged with a stream of N2. It was decanted into an Erlenmeyer flask (most of the Ni remains bound to the magnetic stir bar) before being diluted with CHCl3 (100 mL). The solution was then heated to reflux and hot-filtered through a well-packed bed of Celite. The filter cake was rinsed further with hot CHCl3. The filtrates were combined and concentrated in vacuo to afford the product, which was used directly in the next step. Following the General Saponification Procedure, 1.35 g of the 6-mer-HCA (90% over hydrogenolysis and saponification) was collected as an off-white solid. 1H NMR (500 MHz, THF-d8, 25° C.) δ=8.08 (d, J=8.0 Hz, 2H), 7.36 (s, 2H), 7.28-7.20 (m, 6H), 7.15 (s, 2H), 7.12-7.09 (m, 2H), 2.47 (s, 6H), 2.31 (s, 6H), 2.29 (s, 6H), 2.28 (s, 6H) ppm. 13C NMR (126 MHz, THF-d8, 25° C.) δ=173.6, 163.7, 151.1, 142.8, 142.1, 141.7, 141.2, 134.6, 134.2, 134.1, 133.4, 133.0, 132.9, 132.2, 132.1, 132.0, 131.4, 121.5, 119.3, 112.5, 20.7, 20.6, 20.19, 20.18, 20.1, 20.0 ppm. 1H NMR (600 MHz; CD3SOCD3, 100° C.): δ=7.88 (d, J=7.8 Hz, 2H), 7.18 (s, 2H), 7.09 (s, 2H), 7.09 (s, 2H), 7.06 (s, 2H), 6.97-6.94 (m, 4H), 2.28 (s, 6H), 2.11 (s, 6H), 2.10 (s, 12H) ppm. 13C NMR (151 MHz; CD3SOCD3, 100° C.): δ=171.8, 161.3, 149.2, 141.3, 140.5, 140.1, 139.6, 133.4, 132.88, 132.77, 132.1, 131.8, 131.03, 130.95, 130.93, 130.6, 120.8, 117.9, 112.5, 19.8, 19.38, 19.32, 19.28 ppm. HRMS (ESI) Calcd for C46H42O6: m/z=689.2909 ([M−H]−); Found m/z=689.2912.

7-Mer-BME: Following the General Coupling Procedure, 9 (100 mg, 0.174 mmol, 2.3 equiv) and 1,4-diiodo-2,5-dimethyl benzene[20] (27.0 mg, 0.0754 mmol, 1.0 equiv) were dissolved in a degassed mixture of dioxane (3 mL) and H2O (1.5 mL) and reacted with CsF (68.7 mg, 0.452 mmol, 6.0 equiv) and PdCl2(dppf) (6.2 mg, 10 mol %). After completion of the reaction, the mixture was cooled to rt and the precipitate was filtrated off, washed with H2O and MeOH and dried under vacuum to give the 7-mer-BME as a colorless solid (64 mg, 0.064 mmol, 84%). 1H NMR (500 MHz, CDCl3, 25° C.): δ=7.95 (d, J=8.0 Hz, 2H), 7.56 (m, 4H), 7.44 (m, 4H), 7.35 (m, 2H), 7.15-7.12 (m, 8H), 7.09-7.07 (m, 6H), 5.28 (s, 4H), 3.97 (s, 6H), 2.22 (s, 6H), 2.18-2.15 (m, 24H) ppm. 13C NMR (126 MHz, CDCl3, 25° C.): δ=166.8, 157.9, 147.6, 141.43, 141.36, 140.58, 140.53, 140.47, 140.32, 140.27, 140.23, 140.21, 140.17, 139.82, 139.78, 139.77, 139.73, 139.45, 139.42, 136.8, 133.55, 133.49, 133.02, 132.96, 132.91, 132.89, 132.86, 132.83, 132.81, 132.78, 132.75, 132.72, 132.70, 132.62, 132.59, 132.08, 132.05, 131.72, 131.67, 130.90, 130.87, 130.78, 130.71, 130.64, 128.6, 127.8, 126.8, 121.7, 118.8, 115.1, 70.6, 52.1, 25.1, 19.84, 19.80, 19.54, 19.52, 19.49, 19.47, 19.38, 19.36 ppm. HRMS (ESI): m/z calcd for C70H67O6 [M+H]+ 1003.4932; found 1003.4952.

7-Mer-HCA: The General Hydrogenolysis Procedure, with THF (10 mL), benzene (10 mL), Pd/C (30.0 mg, 0.028 mmol) and the 7-mer-BME (300 mg, 0.299 mmol) were followed. The reaction mixture was stirred for 48 h at 120 psi of H2 at 60° C. It was then cooled to rt, purged with a stream of N2 and filtered. The filter cake was washed with hot PhCl (50 mL) and the combined filtrate was concentrated in vacuo to afford the product which was used directly in the next step. Following the General Saponification Procedure, 237 mg of the 7-mer-HCA were collected as an off-white very poorly soluble solid. Low solubility hindered full characterization. An 1H NMR spectrum in CD3SOCD3 was only obtained after a prolonged acquisition time and at elevated temperature. It did not prove possible to record a 13C NMR spectrum. 1H NMR (600 MHz; CD3SOCD3, 80° C.): δ=7.86 (d, J=7.9 Hz, 2H), 7.17 (s, 2H), 7.08 (m, 6H), 7.05 (s, 2H), 6.95-6.93 (m, 4H), 2.26 (s, 6H), 2.08 (m, 24H) ppm. HRMS (ESI): m/z calcd for C54H49O6 [M−H]− 793.3535; found 793.3525.

Hex-3-mer-BME: Following the General Coupling Procedure, 3 (3.40 g, 9.23 mmol, 2.3 equiv) and 12 (2.00 g, 4.01 mmol, 1.0 equiv) were dissolved in a degassed mixture of dioxane (80 mL) and H2O (40 mL) and reacted with CsF (3.65 g, 24.1 mmol, 6.0 equiv) and PdCl2(dppf) (328 mg, 10 mol %). After completion of the reaction, the mixture was cooled to rt before being extracted with H2O and CH2Cl2. The aqueous phase was washed twice with CH2Cl2. The combined organic phases were dried (MgSO4) and concentrated. The crude product was subjected to column chromatography (SiO2: hexanes:EtOAc=5:1) to give the hex-3-mer-BME as a colorless solid (2.60 g, 3.58 mmol, 89%). 1H NMR (500 MHz, CD2Cl2, 25° C.): δ=7.93 (d, J=8.2 Hz, 2H), 7.53 (d, J=7.5 Hz, 4H), 7.42 (t, J=7.6 Hz, 4H), 7.34 (t, J=7.3 Hz, 2H), 7.07 (s, 2H), 7.01 (m, 4H), 5.25 (s, 4H), 3.97 (s, 6H), 2.49 (t, J=8.0 Hz, 4H), 1.42 (m, 4H), 1.25-1.15 (m, 12H), 0.85 (t, J=7.1 Hz, 6H) ppm. 13C NMR (126 MHz, CD2Cl2, 25° C.): δ=166.7, 157.8, 147.3, 140.3, 137.5, 136.7, 131.7, 130.6, 128.6, 127.8, 126.8, 121.6, 119.0, 115.0, 70.6, 52.1, 32.6, 31.57, 31.47, 29.2, 22.6, 14.1 ppm. HRMS (ESI): m/z calcd for C48H55O6 [M+H]+ 727.3993; found 727.3995.

Hex-3-mer-HCA: Following the General Hydrogenolysis Procedure, with the hex-3-mer-BME (2.20 g, 3.03 mmol, 1.0 equiv), THF (50 mL) and Pd/C (10%, 321 mg), the product was obtained as a colorless solid and used directly in the next step. Following the General Saponification Procedure, the hex-3-mer-HCA was obtained as a colorless solid (1.57 g, 3.03 mmol, quant). 1H NMR (500 MHz, CD3SOCD3, 25° C.): δ=7.85 (d, J=8.4 Hz, 2H), 7.12 (s, 2H), 6.91 (m, 4H), 2.56 (m, 4H), 1.40 (m, 4H), 1.18-1.09 (m, 12H), 0.77 (t, J=7.0 Hz, 6H) ppm. 13C NMR (126 MHz; CD3SOCD3, 25° C.): δ=171.8, 160.9, 148.5, 139.6, 136.9, 130.3, 130.1, 120.2, 117.3, 111.7, 31.7, 30.68, 30.55, 28.4, 21.8, 13.8 ppm. HRMS (ESI): m/z calcd for C32H38O6 [M−H]− 517.2596; found 517.2593.

Hex-5-mer-BME: Following the General Coupling Procedure, 7 (697 mg, 1.48 mmol, 2.2 equiv) 12 (334 mg, 0.670 mmol, 1.0 eq) were dissolved in a degassed mixture of dioxane (15 mL) and H2O (7.5 mL) and reacted with CsF (610 mg, 4.02 mmol, 6.0 equiv) and PdCl2(dppf) (55 mg, 0.067 mmol, 10 mol %). After completion of the reaction, the mixture was extracted with H2O and CH2Cl2. The aqueous phase was washed twice with CH2Cl2. The combined organic phases were dried (MgSO4) and concentrated. The crude product was subjected to column chromatography (SiO2: CH2Cl2:hexanes=1:1˜1:0) to give the hex-5-mer-BME as a colorless solid (465 mg, 0.497 mmol, 74%). 1H NMR (500 MHz, CDCl3, 25° C.): δ=7.95 (d, J=8.2 Hz, 2H), 7.56 (d, J=7.5 Hz, 4H), 7.44 (t, J=7.6 Hz, 4H), 7.35 (t, J=7.4 Hz, 2H), 7.14 (m, 4H), 7.09-7.06 (m, 6H), 5.28 (s, 4H), 3.98 (s, 6H), 2.49 (m, 2H), 2.40-2.35 (m, 2H), 2.22 (s, 6H), 2.15 (d, J=3H), 2.14 (s, 3H), 1.48 (m, 4H), 1.22 (m, 12H), 0.85 (t, J=7.0 Hz, 6H) ppm. 13C NMR (126 MHz, CDCl3, 25° C.): δ=166.8, 157.9, 147.6, 141.34, 141.25, 139.51, 139.49, 139.40, 139.37, 137.41, 137.36, 136.8, 133.64, 133.58, 132.00, 131.93, 131.82, 131.81, 131.66, 130.77, 130.74, 130.06, 130.00, 128.6, 127.8, 126.8, 121.7, 118.8, 115.1, 70.6, 52.1, 32.51, 32.46, 31.6, 30.75, 30.66, 29.00, 28.95, 22.5, 19.85, 19.81, 19.72, 19.58, 14.1 ppm. HRMS (ESI): m/z calcd for C64H71O6 [M+H]+ 935.5245; found 935.5213.

Hex-Smer-HCA: Following the General Hydrogenolysis Procedure, with hex-5-mer-BME (455 mg, 0.467 mmol, 1.0 equiv), THF (10 mL) and Pd/C (10%, 50 mg) the debenzylated compound was obtained and used directly in the next step. Following the General Saponification Procedure, the hex-5-mer-HCA was obtained as a colorless solid (339 mg, 30.467 mmol, quant). 1H NMR (600 MHz, CD3SOCD3, 100° C.).: δ=7.85 (d, J=8.4 Hz, 2H), 7.15 (s, 2H), 7.07 (s, 2H), 7.01 (s, 2H), 6.89 (m, 4H), 2.46 (m, 2H), 2.34 (m, 2H), 2.25 (s, 6H), 2.07 (s, 6H), 1.41 (m, J=7.1 Hz, 4H), 1.15 (m, J=13.9, 7.1 Hz, 12H), 0.78 (t, J=7.1 Hz, 6H) ppm. 13C NMR (126 MHz, CD2Cl2, 25° C.): δ=171.8, 161.5, 148.9, 141.1, 139.79, 139.71, 137.5, 133.4, 132.1, 131.9, 130.9, 130.55, 130.44, 120.4, 117.8, 113.1, 32.5, 31.2, 30.4, 28.6, 22.1, 19.8, 19.4, 14.0 ppm. HRMS (ESI): m/z calcd for C48H53O6 [M−H]− 725.3848; found 725.3834.

Hex-7-mer-BME: Following the General Coupling Procedure, 9 (641 mg, 1.11 mmol, 2.2 equiv) and 12 (252 mg, 0.505 mmol, 1.0 eq) were dissolved in a degassed mixture of dioxane (11 mL) and H2O (5.5 mL) and reacted with CsF (460 mg, 43.03 mmol, 6.0 equiv) and PdCl2(dppf) (41 mg, 0.050 mmol, 10 mol %). After completion of the reaction, the mixture was cooled to rt and extracted with H2O and CH2Cl2. The aqueous phase was washed twice with CH2Cl2. The combined organic phases were dried (MgSO4) and concentrated. The crude product was subjected to column chromatography (SiO2: CH2Cl2) to give the hex-7-mer-BME as a colorless solid (527 mg, 0.460 mmol, 91%). 1H NMR (500 MHz, CDCl3, 25° C.): δ=7.95 (d, J=8.2 Hz, 2H), 7.56 (d, J=7.6 Hz, 4H), 7.44 (t, J=7.6 Hz, 4H), 7.35 (t, J=7.4 Hz, 2H), 7.17-7.06 (m, 15H), 5.28 (s, 4H), 3.98 (s, 6H), 2.51 (m, 2H), 2.39 (m, 2H), 2.22 (s, 6H), 2.18 (s, 3H), 2.15-2.12 (m, 15H), 1.50-1.45 (m, 4H), 1.28-1.17 (m, 12H), 0.85 (m, 6H) ppm. 13C NMR (126 MHz, CDCl3, 25° C.): δ=166.8, 157.9, 147.6, 141.50, 141.40, 140.5, 139.72, 139.64, 139.44, 139.40, 137.56, 137.51, 136.8, 133.55, 133.48, 133.25, 133.20, 133.14, 133.12, 133.02, 132.96, 132.95, 132.45, 132.43, 132.31, 132.09, 132.03, 131.76, 131.67, 131.58, 131.26, 131.21, 131.18, 131.12, 130.77, 130.58, 130.26, 130.17, 130.13, 128.6, 127.8, 126.8, 121.7, 118.8, 115.1, 70.6, 52.1, 32.8, 32.58, 32.53, 31.64, 31.59, 31.48, 31.08, 30.95, 30.85, 30.70, 29.15, 29.10, 29.07, 22.71, 22.58, 22.55, 19.86, 19.79, 19.62, 19.52, 19.49, 19.44, 19.2, 14.16, 14.08 ppm. HRMS (ESI): m/z calcd for C96H103O6 [M+H]+ 1350.7676; found 1351.7733.

Hex-7-mer-HCA: Following the General Hydrogenolysis Procedure with the hex-7-mer-BME (1.19 g, 1.04 mmol, 1.0 equiv) THF (50 mL) and Pd/C (10%, 110 mg), the debenzylated compound was obtained and used directly in the next step. Following the General Saponification Procedure, in THF (130 mL) and 0.5 M aq. NaOH (110 mL), the hex-7-mer-HCA was obtained as a colorless solid (973 mg, 1.04 mmol, quant). 1H NMR (600 MHz, CD3SOCD3, 90° C.): δ=7.86 (d, J=8.0 Hz, 2H), 7.17 (s, 2H), 7.09 (s, 2H), 7.07 (m, 6H), 6.96 (m, 4H), 2.26 (s, 6H), 2.08 (m, 18H), 1.42 (m, 4H), 1.20-1.12 (m, 16H), 0.80 (t, J=6.6 Hz, 6H) ppm. 13C NMR (151 MHz, CD3SOCD3, 90° C.): δ=171.9, 161.3, 149.2, 141.3, 140.5, 140.02, 139.96, 139.6, 137.7, 133.3, 133.0, 132.5, 132.1, 131.7, 131.4, 130.95, 130.88, 130.57, 130.51, 120.8, 117.9, 112.4, 32.6, 31.2, 30.9, 30.6, 29.8, 28.7, 22.2, 19.8, 19.4, 14.0 ppm. HRMS (ESI): m/z calcd for C64H96O6 [M−H]− 934.5172; found 934.5162.

Hex-9-mer-BME: Following the General Coupling Procedure, 11 (916 mg, 1.35 mmol, 2.2 equiv) and 12 (305 mg, 0.612 mmol, 1.0 equiv) were dissolved in a degassed mixture of dioxane (18 mL) and H2O (9 mL) and reacted with CsF (558 mg, 3.67 mmol, 6.0 equiv) and PdCl2(dppf) (50.0 mg, 0.061 mmol, 10 mol %). After completion of the reaction, the mixture was cooled to rt and the precipitate was filtered off, washed with H2O and MeOH, dried under vacuum and subjected to column chromatography (SiO2: CH2Cl2) to give the hex-9-mer-BME as a colorless solid (700 mg, 0.518 mmol, 85%). 1H NMR (500 MHz, CDCl3, 25° C.): δ=7.93 (d, J=8.3 Hz, 2H), 7.54 (d, J=7.2 Hz, 4H), 7.42 (t, J=7.6 Hz, 4H), 7.33 (t, J=7.4 Hz, 2H), 7.17-7.10 (m, 12H), 7.07-7.05 (m, 6H), 5.27 (s, 4H), 3.96 (s, 6H), 2.53-2.37 (m, 4H), 2.20 (s, 6H), 2.14 (m, 30H), 1.48 (m, 4H), 1.21 (m, J=6.7 Hz, 12H), 0.88-0.81 (m, 6H) ppm. 13C NMR (126 MHz, CDCl3, 25° C.): δ=166.8, 157.9, 147.6, 141.45, 141.39, 140.71, 140.63, 140.56, 140.46, 140.37, 140.28, 140.18, 140.13, 140.09, 140.03, 139.93, 139.90, 139.81, 139.76, 139.70, 139.45, 139.43, 137.63, 137.60, 137.56, 137.54, 137.51, 136.8, 133.56, 133.51, 133.10, 133.05, 132.98, 132.94, 132.88, 132.73, 132.67, 132.64, 132.57, 132.53, 132.45, 132.39, 132.08, 132.06, 131.73, 131.68, 131.22, 131.13, 130.98, 130.95, 130.73, 130.65, 130.35, 130.30, 130.22, 130.17, 130.07, 130.03, 128.6, 127.8, 126.8, 121.7, 118.8, 115.1, 70.6, 52.1, 32.90, 32.85, 32.79, 32.67, 32.62, 32.57, 31.61, 31.49, 31.11, 31.02, 30.98, 30.88, 30.86, 30.72, 29.23, 29.18, 29.15, 29.12, 29.10, 29.05, 22.59, 22.57, 19.85, 19.81, 19.78, 19.76, 19.64, 19.60, 19.55, 19.50, 19.47, 19.40, 19.37, 19.33, 19.28, 14.17, 14.09 ppm. HRMS (ESI): m/z calcd for C96H103O6 [M+H]+ 1351.7749; found 1351.7758.

Hex-9-mer-HCA: Following the General Hydrogenolysis Procedure with hex-9-mer-BME (827 mg, 0.612 mmol, 1.0 equiv) and Raney Ni (nmol 10%) THF (50 mL), the debenzylated compound was obtained and used directly in the next step. Following the General Saponification Procedure, the hex-9-mer-HCA was obtained as a colorless solid (700 mg, 0.612 mmol, quant). 1H NMR (600 MHz, CD3SOCD3, 100° C.): δ=7.87 (d, J=8.1 Hz, 2H), 7.17 (s, 2H), 7.10 (s, 2H), 7.08 (m, 8H), 7.05 (s, 2H), 6.94 (m, 4H), 2.27 (s, 6H), 2.09 (m, 30H), 1.44 (m, 4H), 1.26 (m, 4H), 1.22-1.14 (m, 12H), 0.81 (t, J=7.1 Hz, 6H). 13C NMR (151 MHz, THF-d8, 55° C.): δ=170.0, 160.4, 147.9, 139.5, 138.63, 138.55, 138.32, 138.23, 137.9, 131.2, 130.93, 130.86, 130.68, 130.49, 130.0, 129.57, 129.51, 129.2, 128.90, 128.73, 128.71, 128.67, 128.61, 128.0, 123.0, 118.1, 115.9, 109.2, 32.2, 29.62, 29.55, 28.0, 27.7, 27.02, 26.96, 20.5, 17.0, 16.73, 16.68, 16.59, 11.45, 11.38 ppm. HRMS (ESI): m/z calcd for C80H85O6 [M−H]− 1141.6352; found 1141.6295.

13: 1,4-Dibromo-2,5-dimethylbenzene (25.0 g, 94.7 mmol, 1.0 equiv), bis(pinacolato)diboron (50.5 g, 199 mmol, 2.1 equiv), PdCl2(dppf) (3.87 g, 4.74 mmol, 5 mol %), and KOAc (55.8 g, 568 mmol, 6.0 equiv) were added to a flame-dried flask containing anhydrous Me2SO (300 mL). The reaction mixture was heated under reflux for 16 h. It was then cooled to rt, added to H2O (100 mL), before being extracted three times with CH2Cl2. The organic layer was then washed extensively with H2O. The organic phase was dried (MgSO4), filtered, and concentrated. EtOAc (250 mL) was added to the crude mixture, along with activated carbon (10 g) and the reaction mixture was heated under reflux for 3 h. It was cooled to rt, filtered through Celite, and the Celite was washed thoroughly with CH2Cl2. The CH2Cl2 layer was concentrated, causing compound 13 to precipitate from the EtOAc as fine off-white needles, which were isolated by vacuum filtration (14.6 g, 40.8 mmol, 43%). 1H NMR (500 MHz, CDCl3, 25° C.): δ=7.53 (s, 2H), 2.48 (s, 6H), 1.34 (s, 24H) ppm. 13C NMR (126 MHz, CDCl3, 25° C.): δ=140.7, 137.0, 83.5, 25.0, 21.6 ppm. HRMS (ESI): m/z calcd for C20H33B2O4 [M+H]+ 357.2632; found 357.2614.

14: Following the General Coupling Procedure, 13 (5.00 g, 13.9 mmol, 1.0 equiv) and 4 (18.4 g, 69.8 mmol, 5.0 equiv) were dissolved in a degassed 2:1 dioxane/H2O mixture (75 mL) and reacted with CsF (12.7 g, 83.8 mmol, 6.0 equiv) and PdCl2(dppf) (1.14 g, 1.40 mmol, 10 mol %). After completion of the reaction, the mixture was cooled to rt, before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes) to give compound 14 as a colorless solid (2.18 g, 4.61 mmol, 33%). 1H NMR (500 MHz, CD2Cl2, 25° C.): δ=7.47 (s, 2H), 7.04 (m, 2H), 6.95 (s, 2H), 2.39 (s, 6H), 2.05 (m, 6H), 2.02 (s, 6H) ppm. 13C NMR (126 MHz, CD2Cl2, 25° C.): δ=141.2, 139.91, 139.89, 135.9, 135.1, 133.5, 133.2, 133.1, 132.1, 132.0, 130.9, 123.4, 22.5, 22.4, 19.4, 19.3, 19.2 ppm.

15: 14 (1.00 g, 2.12 mmol, 1.0 equiv), bis(pinacolato)diboron (1.13 g, 4.45 mmol, 2.1 equiv), PdCl2(dppf) (173 mg, 0.212 mmol, 10 mol %), and KOAc (1.25 g, 12.7 mmol, 6.0 equiv) were added to a flame-dried flask containing anhydrous Me2SO (50 mL) and the reaction mixture was heated under reflux for 16 h. It was cooled to rt, added to H2O (25 mL), and then extracted three times with CH2Cl2. The organic layer was then washed extensively with H2O. The organic phase was dried (MgSO4), filtered, and concentrated. The crude product was absorbed on silica-gel and subjected to column chromatography (SiO2: hexanes) to give compound 15 as a colorless solid (371 mg, 0.655 mmol, 31%). 1H NMR (500 MHz, CD2Cl2, 25° C.): δ=7.66 (s, 2H), 6.99 (m, 4H), 2.54 (s, 6H), 2.10 (s, 6H), 2.04 (s, 6H), 1.39 (s, 24H) ppm. 13C NMR (126 MHz, CD2Cl2, 25° C.): δ=144.5, 142.2, 140.7, 137.7, 132.9, 132.8, 132.4, 131.3, 131.2, 130.7, 83.8, 25.1, 21.80, 21.78, 19.4, 19.3, 19.1 ppm. HRMS (ESI): m/z calcd for C36H49B2O4 [M+H]+ 565.3884; found 565.3864.

16: Following the General Coupling Procedure, 9 (1.50 g, 2.61 mmol, 1.0 equiv) and 12 (5.20 g, 10.4 mmol, 4.0 equiv) were dissolved in a degassed mixture of dioxane (100 mL) and H2O (50 mL) at 80° C. and reacted with CsF (1.19 g, 7.83 mmol, 3.0 equiv) and PdCl2(dppf) (106 mg, 0.131 mmol, 5 mol %). After completion of the reaction, the mixture was cooled to rt and extracted with CH2Cl2 and H2O. The aqueous phase was washed twice with CH2Cl2, the combined organic phases were dried (MgSO4), concentrated, absorbed on silica-gel and subjected to column chromatography (SiO2: hexanes:EtOAc=5:1) in order to afford compound 16 as a colorless solid (856 mg, 1.04 mmol, 40%). 1H NMR (500 MHz, CDCl3, 25° C.): δ=7.94 (d, J=8.3 Hz, 1H), 7.76 (s, 1H), 7.55 (d, J=7.2 Hz, 2H), 7.43 (t, J=7.6 Hz, 2H), 7.35 (t, J=7.4 Hz, 1H), 7.13 (s, 1H), 7.10-7.00 (m, 6H), 5.28 (s, 2H), 3.98 (s, 3H), 2.72 (m, 2H), 2.42 (m, 1H), 2.31 (m, 1H), 2.21 (s, 3H), 2.15-2.11 (m, 6H), 2.06 (m, 3H), 1.46-1.14 (m, 16H), 0.93-0.83 (m, 6H) ppm. 13C NMR (126 MHz, CDCl3, 25° C.): δ=166.8, 157.9, 147.5, 142.3, 141.39, 141.26, 140.55, 140.52, 140.13, 140.08, 139.61, 139.53, 139.48, 139.36, 139.27, 136.8, 133.45, 133.37, 132.82, 132.67, 132.54, 132.13, 132.07, 131.67, 131.47, 130.90, 130.84, 130.78, 130.69, 130.50, 130.40, 128.6, 127.8, 126.8, 121.7, 118.8, 115.1, 99.2, 70.6, 52.1, 40.38, 40.34, 34.7, 32.4, 32.1, 31.7, 31.45, 31.36, 30.81, 30.66, 30.43, 30.30, 29.15, 29.08, 29.00, 25.3, 22.69, 22.50, 19.84, 19.77, 19.62, 19.47, 19.2, 14.15, 14.06 ppm. HRMS (ESI): m/z calcd for C49H58IO3 [M+H]+ 821.3425; found 821.3424.

Hex-11-mer-BME: Following the General Coupling Procedure, 15 (151 mg, 0.266 mmol, 1.0 equiv) and 16 (480 mg, 0.585 mmol, 2.2 equiv) were suspended in a degassed mixture of dioxane (12 mL) and H2O (6 mL) and reacted with CsF (242 mg, 1.60 mmol, 6.0 equiv) and PdCl2(dppf) (22 mg, 0.0269 mmol, 10 mol %). After completion of the reaction, the mixture was cooled to rt and the precipitate was filtered off, washed with H2O and MeOH and dried at vacuum. The beige crude product was subjected to column chromatography (SiO2: CH2Cl2) to yield the hex-11-mer-BME as a colorless solid (373 mg, 0.219 mmol, 82%). 1H NMR (500 MHz, CDCl3, 25° C.): δ=7.96 (d, J=8.2 Hz, 2H), 7.57 (d, J=7.6 Hz, 4H), 7.44 (t, J=7.6 Hz, 4H), 7.36 (t, J=7.4 Hz, 2H), 7.19-7.07 (m, 22H), 5.29 (s, 4H), 3.99 (s, 6H), 2.54 (m, 4H), 2.41 (m, 4H), 2.23-2.14 (m, 46H), 1.54-1.46 (m, 8H), 1.28-1.17 (m, 24H), 0.87 (m, 12H) ppm. 13C NMR (126 MHz, CDCl3, 25° C.): δ=166.8, 157.9, 147.6, 141.53, 141.43, 140.68, 140.65, 140.57, 140.47, 140.33, 140.28, 140.21, 140.18, 140.02, 139.99, 139.90, 139.87, 139.78, 139.71, 139.68, 139.63, 139.60, 139.58, 139.45, 139.40, 137.67, 137.64, 137.61, 137.57, 137.54, 137.51, 137.49, 137.46, 137.44, 136.8, 133.57, 133.50, 133.21, 133.15, 133.05, 132.99, 132.87, 132.82, 132.76, 132.71, 132.66, 132.64, 132.60, 132.51, 132.50, 132.45, 132.42, 132.31, 132.09, 132.04, 131.78, 131.68, 131.60, 131.29, 131.24, 131.20, 131.16, 131.11, 130.90, 130.78, 130.71, 130.67, 130.59, 130.37, 130.32, 130.25, 130.19, 130.13, 130.10, 130.06, 130.04, 130.00, 128.6, 127.8, 121.7, 118.8, 115.1, 70.6, 52.1, 34.7, 32.8, 32.62, 32.61, 31.61, 31.50, 31.10, 31.01, 30.97, 30.87, 30.84, 30.72, 29.23, 29.18, 29.14, 29.10, 29.05, 22.60, 22.57, 20.8, 19.87, 19.80, 19.78, 19.64, 19.61, 19.53, 19.50, 19.48, 19.46, 19.37, 19.31, 19.25, 14.17, 14.09 ppm. HRMS (ESI): m/z calcd for C122H139O6 [M+H]+ 1700.0566; found 1700.0582.

Hex-11-mer-HCA: Following the General Hydrogenolysis Procedure, the hex-11-mer-BME (372 mg, 0.219 mmol, 1.0 equiv) and Raney Ni (≈mol 10%) in THF (30 mL) provided the debenzylated compound which was used directly in the next step. Following the General Saponification Procedure provided the hex-11-mer-HCA as a colorless solid (326 mg, 0.219 mmol, quant). 1H NMR (600 MHz, CD3SOCD3, 100° C.): δ=7.87 (d, J=7.8 Hz, 2H), 7.17 (s, 2H), 7.11-7.05 (m, 16H), 6.95 (m, 4H), 2.27 (s, 6H), 2.11-2.09 (m, 36H), 1.44 (m, 8H), 1.26-1.15 (m, 32H), 0.81 (m, 12H) ppm. 1H NMR (600 MHz, THF, 56° C.): δ=7.92 (d, J=8.0 Hz, 2H), 7.18 (s, 2H), 7.16 (s, 2H), 7.12 (t, J=9.7 Hz, 12H), 7.07 (s, 2H), 6.98 (s, 2H), 6.92 (d, J=7.8 Hz, 2H), 2.56 (m, 4H), 2.45 (m, 4H), 2.30 (s, 6H), 2.16 (s, 18H), 2.15 (s, 12H), 2.13 (s, 6H), 1.53 (m, 8H), 1.26 (s, 24H), 0.87 (m, J=7.6 Hz, 12H) ppm. 13C NMR (151 MHz, THF-d8, 55° C.): δ=171.9, 162.2, 149.7, 141.3, 140.61, 140.46, 140.1, 139.8, 137.6, 137.2, 133.12, 133.09, 132.85, 132.78, 132.65, 132.3, 131.9, 131.45, 131.29, 131.14, 131.09, 131.04, 130.74, 130.66, 130.56, 130.48, 130.31, 130.20, 130.06, 129.86, 124.9, 119.9, 117.8, 111.1, 32.8, 32.6, 31.48, 31.41, 30.90, 30.82, 30.68, 29.8, 29.6, 29.05, 28.95, 22.35, 22.33, 18.98, 18.89, 18.86, 18.76, 18.51, 18.47, 18.39, 13.32, 13.25 ppm. HRMS (ESI): m/z calcd for C106H123O6 [M+H]+ 1491.9314; found 1491.9260. m/z calcd for C106H121O6 [M−H]− 1489.9169; found 1489.9145.

18: Methyl 4-bromo-3-methylbenzoate (17) (5.00 g, 21.8 mmol, 1.0 equiv) and bis(pinacolato)diboron (6.10 g, 24.0 mmol, 1.1 equiv) were dissolved in anhydrous degassed Me2SO (80 mL). KOAc (6.42 g, 65.4 mmol, 3.0 equiv) and PdCl2(dppf) (890 mg, 1.09 mmol, 5 mol %) were added and the reaction mixture was heated to 80° C. overnight, before being cooled to rt and extracted with CH2Cl2 and H2O. The aqueous phase was washed twice with CH2Cl2. The combined organic phases were washed with H2O, dried (MgSO4), concentrated and subjected to column chromatography (SiO2: hexanes:EtOAc=5:1) to give compound 18 as a colorless oil (5.84 g, 21.2 mmol, 97%). 1H NMR (500 MHz, CDCl3, 25° C.): =7.81 (m, 3H), 3.91 (s, 3H), 2.57 (s, 3H), 1.35 (s, 12H) ppm. 13C NMR (126 MHz, CDCl3, 25° C.): =167.4, 144.9, 135.8, 131.8, 130.5, 125.6, 83.9, 52.1, 24.9, 22.1 ppm. HRMS (ESI): m/z calcd for C15H22BO4 [M+H]+ 277.1608; found 277.1611.

19: Following the General Coupling Procedure, 18 (1.42 g, 5.14 mmol, 1.0 equiv) and 4 (6.79 g, 25.7 mmol, 5.0 equiv) were dissolved in a degassed mixture of dioxane (500 mL) and H2O (200 mL) at 80° C. before CsF (2.34 g, 15.4 mmol, 3.0 equiv) and PdCl2(dppf) (210 mg, 0.260 mmol, 5 mol %) were added. After completion of the reaction, the mixture was cooled to rt, before being extracted with CH2Cl2 and H2O. The aqueous phase was washed twice with CH2Cl2. The combined organic phases were washed with brine, dried (MgSO4) and concentrated. The crude product was subjected to column chromatography (SiO2: hexanes:EtOAc=10:1) to give the product as a colorless oil (973 mg, 2.93 mmol, 57%). 1H NMR (500 MHz, CDCl3, 25° C.): =7.97 (s, 1H), 7.97 (s, 1H), 7.91 (dd, J=7.9, 1.3 Hz, 1H), 7.91 (dd, J=7.9, 1.3 Hz, 1H), 7.48 (s, 1H), 7.48 (s, 1H), 7.16 (d, J=7.9 Hz, 1H), 7.16 (d, J=7.9 Hz, 1H), 6.97 (s, 1H), 6.97 (s, 1H), 3.96 (s, 3H), 3.96 (s, 3H), 2.40 (s, 3H), 2.40 (s, 3H), 2.12 (s, 3H), 2.12 (s, 3H), 1.99 (s, 3H), 1.99 (s, 3H) ppm. 13C NMR (126 MHz, CDCl3, 25° C.): =167.2, 145.4, 139.8, 136.2, 135.05, 134.85, 133.5, 131.09, 130.98, 129.40, 129.23, 126.9, 123.8, 52.2, 22.4, 19.8, 19.0 ppm. HRMS (ESI): m/z calcd for C17H18Br2 [M+H]+ 333.0485; found 333.0479.

20: Compound 19 (900 mg, 2.70 mmol, 1.0 equiv) and bis(pinacolato)diboron (754 mg, 2.97 mmol, 1.1 equiv) were dissolved in anhydrous degassed Me2SO (11 mL). KOAc (795 mg, 8.10 mmol, 3.0 equiv) and PdCl2(dppf) (110 mg, 0.135 mmol, 5 mol %) were added and the reaction mixture was heated to 80° C. overnight, before being cooled to rt and extracted with CH2Cl2 and H2O. The aqueous phase was washed twice with CH2Cl2. The combined organic phases were washed with H2O, dried (MgSO4), concentrated and subjected to column chromatography (SiO2: hexanes:EtOAc=5:1) to give compound 20 as a colorless oil (880 mg, 2.38 mmol, 88%). 1H NMR (500 MHz, CDCl3, 25° C.): =7.97 (s, 1H), 7.91 (dd, J=7.9, 1.4 Hz, 1H), 7.70 (s, 1H), 7.18 (d, J=7.9 Hz, 1H), 6.92 (s, 1H), 3.96 (s, 3H), 2.55 (s, 3H), 2.12 (s, 3H), 2.02 (s, 3H), 1.39 (s, 12H) ppm. 13C NMR (126 MHz, CDCl3, 25° C.): =167.3, 146.6, 143.2, 142.1, 137.4, 136.1, 131.6, 131.0, 130.2, 129.3, 128.9, 126.8, 83.5, 52.1, 34.7, 25.0, 21.7, 19.8, 19.0 ppm. HRMS (ESI): m/z calcd for C23H30BO4 [M+H]+ 380.2268; found 380.2263.

2-mer-ME: Following the General Coupling Procedure, 18 (830 mg, 3.01 mmol, 1.0 equiv) and 17 (690 mg, 3.01 mmol, 1.0 equiv) were reacted in a degassed 2:1 dioxane/H2O mixture (90 mL) with CsF (1.37 g, 9.03 mmol, 3.0 equiv) and PdCl2(dppf) (123 mg, 0.150 mmol, 5 mol %) for 15 h. The reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=5:1) to give the 2-mer-ME as a colorless solid (502 mg, 1.68 mmol, 56%). 1H NMR (500 MHz, CDCl3, 25° C.): δ=7.99 (s, 2H), 7.93 (dd, J=7.9, 1.5 Hz, 2H), 7.18 (d, J=7.9 Hz, 2H), 3.97 (s, 6H), 2.10 (s, 6H) ppm. 13C NMR (126 MHz, CDCl3, 25° C.): =167.1, 145.5, 135.9, 131.2, 129.5, 129.0, 127.0, 52.2, 19.7 ppm. HRMS (ESI): m/z calcd for C18H19O4 [M+H]+ 299.1278; found 299.1288.

3-mer-ME: Following the General Coupling Procedure, 18 (2.00 g, 7.24 mmol, 2.3 equiv) and 1,4-diiodo-2,5-dimethyl benzene[20] (1.13 g, 3.15 mmol, 1.0 equiv) were reacted in a degassed 2:1 dioxane/H2O mixture (150 mL) with CsF (2.87 g, 18.9 mmol, 3.0 equiv) and PdCl2(dppf) (260 mg, 0.315 mmol, 5 mol %) for 15 h. The reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=9:1) to give the 3-mer-ME as a colorless solid (904 mg, 2.25 mmol, 71%). 1H NMR (500 MHz, CDCl3, 25° C.): δ=8.00 (s, 2H), 7.94 (d, J=7.9 Hz, 2H), 7.11 (m, 1H), 7.00 (d, J=6.9 Hz, 2H), 3.97 (s, 6H), 2.19 (m, J=5.9 Hz, 3H), 2.12 (m, J=6.8 Hz, 3H), 2.05-2.04 (m, 6H) ppm. 13C NMR (126 MHz, CDCl3, 25° C.): δ=167.31, 167.30, 146.44, 146.38, 145.5, 139.90, 139.86, 136.44, 136.35, 135.9, 132.62, 132.53, 131.19, 131.03, 130.3, 129.66, 129.57, 129.45, 129.02, 129.00, 128.96, 127.01, 126.89, 126.84, 52.2, 19.94, 19.80, 19.72, 19.2 ppm. HRMS (ESI): m/z calcd for C26H27O4 [M+H]+ 403.1904; found 403.1903.

4-mer-ME: Following the General Coupling Procedure, 18 (2.00 g, 7.24 mmol, 2.3 equiv) and 5 (1.46 g, 3.15 mmol, 1.0 equiv) were reacted in a degassed 2:1 dioxane/H2O mixture (150 mL) with CsF (2.87 g, 18.9 mmol, 3.0 equiv) and PdCl2(dppf) (260 mg, 0.315 mmol, 5 mol %) for 15 h. The reaction mixture was cooled to rt, before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: hexanes:EtOAc=9:1) to give the 4-mer-ME as a colorless solid (940 mg, 1.86 mmol, 59%). 1H NMR (500 MHz, CDCl3, 25° C.): δ=8.01 (s, 2H), 7.94 (d, J=7.9 Hz, 2H), 7.30 (m, 2H), 7.11-7.09 (m, 2H), 7.01 (d, J=7.1 Hz, 2H), 3.98 (s, 6H), 2.21 (m, J=6.8 Hz, 6H), 2.12 (m, J=6.1 Hz, 6H), 2.05 (m, 6H) ppm. 13C NMR (126 MHz, CDCl3, 25° C.): δ=167.3, 146.71, 146.63, 140.71, 140.66, 140.61, 139.43, 139.40, 139.38, 139.34, 136.49, 136.44, 133.14, 133.04, 132.95, 132.39, 132.34, 132.29, 132.24, 131.19, 131.00, 130.91, 130.85, 130.1, 129.75, 129.65, 129.02, 128.91, 128.88, 126.86, 126.81, 52.1, 19.98, 19.84, 19.48, 19.47, 19.32, 19.28, 19.24 ppm. HRMS (ESI): m/z calcd for C34H35O4 [M+H]+ 507.2530; found 507.2539.

5-mer-ME: Following the General Coupling Procedure, 20 (290 mg, 0.763 mmol, 2.2 equiv) and 1,4-diiodo-2,5-dimethyl benzene[20] (124 mg, 0.347 mmol, 1.0 equiv) were reacted in a degassed 2:1 dioxane/H2O mixture (24 mL) with CsF (316 mg, 2.08 mmol, 3.0 equiv) and PdCl2(dppf) (28.0 mg, 0.035 mmol, 5 mol %) for 15 h. The reaction mixture was cooled to rt before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: CH2Cl2:hexanes=1:1˜1:0) to give the 5-mer-ME as a colorless solid (170 mg, 0.278 mmol, 80%). 1H NMR (500 MHz, tol-d8, 25° C.): δ=8.16 (m, 2H), 8.08-8.04 (m, 2H), 7.16 (m, 4H), 7.11 (m, 2H), 6.98 (m, 2H), 3.63 (m, 6H), 2.19 (m, 6H), 2.15 (m, 6H), 2.08 (m, 6H), 2.02 (m, 6H) ppm. 13C NMR (126 MHz, CDCl3, 25° C.): δ=167.4, 146.77, 146.70, 140.93, 140.88, 140.82, 140.17, 140.14, 139.29, 139.27, 136.52, 136.46, 133.20, 133.14, 133.09, 133.03, 132.90, 132.86, 132.81, 132.79, 132.77, 132.75, 132.71, 132.67, 132.32, 132.29, 132.21, 132.18, 131.00, 130.93, 130.74, 130.67, 130.1, 129.77, 129.67, 128.88, 128.85, 126.85, 126.80, 52.1, 19.98, 19.84, 19.50, 19.46, 19.35, 19.29, 19.24 ppm. HRMS (ESI): m/z calcd for C42H43O4 [M+H]+ 611.3156; found 611.3160.

6-mer-ME: Following the General Coupling Procedure, 20 (290 mg, 0.763 mmol, 2.2 equiv) and 5 (160 mg, 0.347 mmol, 1.0 equiv) were reacted in a degassed 2:1 dioxane/H2O mixture (24 mL) with CsF (316 mg, 2.08 mmol, 3.0 equiv) and PdCl2(dppf) (28.0 mg, 0.035 mmol, 5 mol %) for 15 h. The reaction mixture was cooled to rt, before being extracted with CH2Cl2 and H2O. The aqueous phase was washed twice with CH2Cl2. The combined organic phases were dried (MgSO4), filtered and concentrated. The crude product was absorbed on silica-gel and subjected to column chromatography (SiO2: CH2Cl2:hexanes=1:1˜1:0) to give the 6-mer-ME as a colorless solid (196 mg, 0.274 mmol, 79%). 1H NMR (500 MHz, tol-d8, 25° C.): δ=8.48 (m, 2H), 8.38 (m, 2H), 7.54-7.48 (m, 6H), 7.43 (m, 2H), 7.31 (m, 2H), 3.96 (m, 6H), 2.54-2.51 (m, 12H), 2.48-2.47 (m, 6H), 2.40 (m, 6H), 2.35-2.33 (m, 6H) ppm. 13C NMR (126 MHz, CDCl3, 25° C.): δ=167.4, 146.79, 146.71, 140.93, 140.88, 140.40, 140.35, 140.12, 140.07, 139.30, 139.27, 139.25, 136.52, 136.47, 133.2, 132.86, 132.80, 132.75, 132.72, 132.31, 132.29, 132.20, 132.17, 130.95, 130.93, 130.84, 130.74, 130.71, 130.63, 130.0, 129.77, 129.68, 128.88, 128.85, 126.85, 126.80, 52.1, 19.98, 19.84, 19.51, 19.50, 19.35, 19.29, 19.24 ppm. HRMS (ESI): m/z calcd for C50H51O4 [M+H]+ 715.3782; found 715.3791.

Compound 27: The boronic ester 9 (600 mg, 1.04 mmol, 2.2 eq.) and the dibromide 26 (265 mg, 0.473 mmol, 1.0 eq.) were dissolved in a degassed mixture of dioxane (10 ml) and H2O (5 ml). PdCl2(dppf) (38.4 mg, 0.047 mmol, 10 mol %) and CsF (431 mg, 2.84 mmol, 6.0 eq.) were added and the reaction mixture was heated to 106° C. for 16 h. It was then cooled to rt before being extracted with CH2Cl2 and H2O. The aqueous phase was washed twice with CH2Cl2. The combined organic phases were dried (MgSO4), filtrated and evaporated. The crude product was absorbed on silica-gel and subjected to column chromatography (SiO2: hexanes:EtOAc=1:1) to give the compound 27 as a colourless solid (485 mg, 0.373 mmol, 79%). 1H NMR (500 MHz, CDCl3, 25° C.): δ=7.95 (d, J=8.2 Hz, 2H), 7.56 (d, J=7.6 Hz, 4H), 7.44 (t, J=7.6 Hz, 4H), 7.35 (t, J=7.4 Hz, 2H), 7.21 (s, 2H), 7.14 (s, 2H), 7.11 (s, 2H), 7.08-7.06 (m, 6H), 6.91 (s, 2H), 5.28 (s, 4H), 4.09 (m, 4H), 3.98 (s, 6H), 3.75 (m, 4H), 3.62 (m, 4H), 3.59 (s, 8H), 3.55 (m, 4H), 2.26 (d, 6H), 2.22 (s, 6H), 2.16 (2, 12H) ppm. 13C NMR (126 MHz, CDCl3, 25° C.): δ=166.8, 157.9, 150.0, 147.6, 141.3, 140.0, 139.5, 137.4, 136.8, 134.0, 133.4, 132.5, 132.1, 131.68, 131.64, 130.86, 130.79, 130.5, 128.6, 127.8, 126.8, 121.7, 118.8, 116.1, 115.1, 71.9, 70.87, 70.79, 70.63, 70.55, 69.8, 69.2, 59.1, 52.1, 19.84, 19.72, 19.49, 19.44 ppm. HRMS (ESI): m/z calcd for C82H91O14 [M+H]+ 1299.6403; found 1299.6454.

Link VII-oeg: The starting material 27 (590 mg, 0.454 mmol, 1.0 eq.) was dissolved in THF (40 mL). Raney Ni (˜mol 10%) was added. The reaction mixture was then stirred under an atmosphere of H2 overnight at 50° C. It was then filtrated through a Celite plug, washed with THF, evaporated and dried at vacuum.

The residue was then dissolved in THF (20 mL) and H2O (20 mL) and NaOH (390 mg, 10.0 mmol, 22 eq.) was added. The reaction mixture was stirred at 50° C. overnight. The THF was evaporated and the mixture was acidified to pH 1 with conc. HCl. The precipitate was filtrated off, washed with H2O and dried to give VII-oeg as a colourless solid (495 mg, 0.454 mmol, quant.). 1H NMR (500 MHz; CD3SOCD3, 25° C.): δ=7.87 (d, J=8.5 Hz, 2H), 7.20 (d, J=6.6 Hz, 4H), 7.08 (s, 2H), 7.02 (s, 2H), 6.98 (m, 4H), 6.92 (s, 2H), 4.07 (m, 4H), 3.62 (m, 4H), 3.46-3.36 (m, 16H), 2.27 (s, 6H), 2.19 (s, 6H), 2.09 (s, 6H), 2.08 (s, 6H) ppm. 13C NMR (126 MHz, CD3SOCD3, 25° C.): δ=171.8, 160.9, 149.3, 148.5, 140.7, 139.4, 138.8, 137.2, 133.5, 132.9, 131.80, 131.67, 131.44, 131.33, 130.6, 130.13, 130.06, 129.89, 120.3, 117.3, 115.4, 111.6, 71.2, 69.98, 69.87, 69.6, 69.0, 68.6, 58.0, 19.6, 19.3, 19.11, 19.02 ppm. HRMS (ESI): m/z calcd for C66H75O14 [M+H]+ 935.5245; found 935.5213.

### REFERENCES

[1] a) G. M. Whitesides, J. P. Mathias, C. T. Seto, Science 1991, 254, 1312-1319; b) G. M. Whitesides, B. Grzybowski, Science 2002, 295, 2418-2421.
[2] a) D. H. Busch, N. A. Stephenson, Coord. Chem. Rev. 1990, 100, 119-154; b) S. Anderson, H. L. Anderson, J. K. M. Sanders, Acc. Chem. Res. 1993, 26, 469-475; c) T. J. Hubin, D. H. Busch, Coord. Chem. Rev. 2000, 200, 5-52; d) M.-J. Blanco, J.-C. Chambron, M.-C. Jimenez, J.-P. Sauvage, Top. Stereochem. 2003, 23, 125-173; e) C. A. Schalley, T. Weilandt, J. Bruggemann, F. Vögtle, Top. Curr. Chem. 2004, 248, 141-200; f) F. Aricô, J. D. Badjić, S. J. Cantrill, A. H. Flood, K. C.-F. Leung, Y. Liu, J. F. Stoddart, Top. Curr. Chem. 2005, 249, 201-259; g) M. S. Vickers, P. D. Beer, Chem. Soc. Rev. 2007, 36, 211-211.
[3] a) M. C. T. Fyfe, J. F. Stoddart, Acc. Chem. Res. 1997, 30, 393-401; b) S. J. Cantrill, A. R. Pease, J. F. Stoddart, J. Chem. Soc. Dalton Trans. 2000, 3715-3734; c) Q. Li, W. Zhang, O. \check{S}. Miljanić, C.-H. Sue, Y.-L. Zhao, L. Liu, C. B. Knobler, J. F. Stoddart, O. M. Yaghi, Science 2009, 325, 855-859.
[4] a) D. Philp, J. F. Stoddart, Angew. Chem., Int. Ed. 1996, 35, 1154-1196; b) K. E. Griffiths, J. F. Stoddart, Pure Appl. Chem. 2008, 80, 485-506; c) J. F. Stoddart, H. M. Colquhoun, Tetrahedron 2008, 64, 8231-8263.
[5] a) A. Müller, E. Krickemeyer, H. Bogge, M. Schmidtmann, F. Peters, Angew. Chem., Int. Ed. 1998, 37, 3359-3363; b) D. K. Chand, K. Biradha, M. Fujita, S. Sakamoto, K. Yamaguchi, Chem. Commun. 2002, 2486-2487; c) M. Tominaga, K. Suzuki, M. Kawano, T. Kusukawa, T. Ozeki, S. Sakamoto, K. Yamaguchi, M. Fujita, Angew. Chem., Int. Ed. 2004, 43, 5621-5625; d) M. Fujita, M. Tominaga, A. Hori, B. Therrien, Acc. Chem. Res. 2005, 38, 369-378.
[6] a) S. Leininger, B. Olenyuk, P. J. Stang, Chem. Rev. 2000, 100, 853-908; b) Y. Inokuma, T. Arai, M. Fujita, Nat. Chem. 2010, 2, 780-783; c) A. Granzhan, T. Riis-Johannessen, R. Scopelliti, K. Severin, Angew. Chem., Int. Ed. 2010, 49, 5515-5518; d) A. Granzhan, C. Schouwey, T. Riis-Johannessen, R. Scopelliti, K. Severin, J. Am. Chem. Soc. 2011, 133, 7106-7115; e) V. Balzani, M. Clemente-León, A. Credi, J. N. Lowe, J. D. Badjić, J. F. Stoddart, D. J. Williams, Chem. Eur. J. 2003, 9, 5348-5360; f) B. H. Northrop, F. Aricô, N. Tangchiavang, J. D. Badjić, J. F. Stoddart, Org. Lett. 2006, 8, 3899-3902; g) J. D. Badjić, S. J. Cantrill, R. H. Grubbs, E. N. Guidry, R. Orenes, J. F. Stoddart, Angew. Chem., Int. Ed. 2004, 43, 3273-3278.
[7] a) M. M. Conn, J. Rebek, Chem. Rev. 1997, 97, 1647-1668; b) R. Wyler, J. de Mendoza, J. Rebek Jr, Angew. Chem., Int. Ed. 1993, 32, 1699-1701; c) B. Olenyuk, J. A. Whiteford, A. Fechtenkotter, P. J. Stang, Nature 1999, 398, 796-799; d) B. Olenyuk, M. D. Levin, J. A. Whiteford, J. E. Shield, P. J. Stang, J. Am. Chem. Soc. 1999, 121, 10434-10435; e) T. Heinz, D. M. Rudkevich, J. Rebek, Nature 1998, 394, 764-766; f) N. Takeda, K. Umemoto, K. Yamaguchi, M. Fujita, Nature 1999, 398, 794-796; g) B. Chatterjee, J. C. Noveron, M. J. E. Resendiz, J. Liu, T. Yamamoto, D. Parker, M. Cinke, C. V. Nguyen, A. M. Arif, P. J. Stang, J. Am. Chem. Soc. 2004, 126, 10645-10656; h) A. C. McKinlay, B. Xiao, D. S. Wragg, P. S. Wheatley, I. L. Megson, R. E. Morris, J. Am. Chem. Soc. 2008, 130, 10440-10444.
[8] a) S. R. Batten, R. Robson, Angew. Chem., Int. Ed. 1998, 37, 1460-1494; b) H. Li, M. Eddaoudi, M. O'Keeffe, O. M. Yaghi, Nature 1999, 402, 276-279; c) M. Eddaoudi, D. B. Moler, H. Li, B. Chen, T. M. Reineke, M. O'Keeffe, O. M. Yaghi, Acc. Chem. Res. 2001, 34, 319-330; d) S. Kitagawa, R. Kitaura, S, Noro, Angew. Chem., Int. Ed. 2004, 43, 2334-2375; e) G. Férey, C. Mellot-Draznieks, C. Serre, F. Millange, J. Dutour, S. Surble, I. Margiolaki, Science 2005, 309, 2040-2042; f) R. Matsuda, R. Kitaura, S. Kitagawa, Y. Kubota, R. V. Belosludov, T. C. Kobayashi, H. Sakamoto, T. Chiba, M. Takata, Y. Kawazoe, Y. Mita, Nature 2005, 436, 238-241; g) A. G. Wong-Foy, O. Lebel, A. J. Matzger, J. Am. Chem. Soc. 2007, 129, 15740-15741; h) S. Ma, J. Eckert, P. M. Forster, J. W. Yoon, Y. K. Hwang, J.-S. Chang, C. D. Collier, J. B. Parise, H.-C. Zhou, J. Am. Chem. Soc. 2008, 130, 15896-15902; i) D. Britt, H. Furukawa, B. Wang, T. G. Glover, O. M. Yaghi, Proc. Natl. Acad. Sci. U.S.A. 2009, 106, 20637-20640; j) H. Deng, C. J. Doonan, H. Furukawa, R. B. Ferreira, J. Towne, C. B. Knobler, B. Wang, O. M. Yaghi, Science 2010, 327, 846-850; k) R. A. Smaldone, R. S. Forgan, H. Furukawa, J. J. Gassensmith, A. M. Z. Slawin, O. M. Yaghi, J. F. Stoddart, Angew. Chem., Int. Ed. 2010, 49, 8630-8634.
[9] a) M. Eddaoudi, J. Kim, J. B. Wachter, H. K. Chae, M. O'Keeffe, O. M. Yaghi, J. Am. Chem. Soc. 2001, 123, 4368-4369; b) B. Moulton, J. Lu, A. Mondal, M. J. Zaworotko, Chem. Commun. 2001, 863-864; c) H. Furukawa, J. Kim, K. E. Plass, O. M. Yaghi, J. Am. Chem. Soc. 2006, 128, 8398-8399.
[10] a) A. P. Côté, A. I. Benin, N. W. Ockwig, M. O'Keeffe, A. J. Matzger, O. M. Yaghi, Science 2005, 310, 1166-1170; b) H. M. El-Kaderi, J. R. Hunt, J. L. Mendoza-Cortes, A. P. Côté, R. E. Taylor, M. O'Keeffe, O. M. Yaghi, Science 2007, 316, 268-272; c) R. W. Tilford, W. R. Gemmill, H.-C. zur Loye, J. J. Lavigne, Chem. Mater. 2006, 18, 5296-5301; d) A. P. Côté, H. M. El-Kaderi, H. Furukawa, J. R. Hunt, O. M. Yaghi, J. Am. Chem. Soc. 2007, 129, 12914-12915; e) S. Wan, J. Guo, J. Kim, H. Ihee, D. Jiang, Angew. Chem., Int. Ed. 2008, 47, 8826-8830; f) R. W. Tilford, S. J. Mugavero Iii, P. J. Pellechia, J. J. Lavigne, Adv. Mater. 2008, 20, 2741-2746; g) S. Wan, J. Guo, J. Kim, H. Ihee, D. Jiang, Angew. Chem., Int. Ed. 2009, 48, 5439-5442; h) E. L. Spitler, W. R. Dichtel, Nat. Chem. 2010, 2, 672-677; i) M. Dogru, A. Sonnauer, A. Gavryushin, P. Knochel, T. Bein, Chem. Commun. 2011, 47, 1707-1707; j) S. Wan, F. Gándara, A. Asano, H. Furukawa, A. Saeki, S. K. Dey, L. Liao, M. W. Ambrogio, Y. Y. Botros, X. Duan, S. Seki, J. F. Stoddart, O. M. Yaghi, Chem. Mater., In press.
[11] D. L. Caulder, K. N. Raymond, Acc. Chem. Res. 1999, 32, 975-982.
[12] Besides controlling the size of shape of spheres, cages, capsules, MOFs, COFs MOPs, it is also desired to find building blocks directing the structures of, to name a few: a) Blockcopolymers: F. S. Bates, G. H. Fredrickson, Annu. Rev. Phys. Chem. 1990, 41, 525-557; b) Dendrimers: S. M. Grayson, J. M. J. Fréchet, Chem. Rev. 2001, 101, 3819-3867; c) Membranes: D. E. Discher, A. Eisenberger, Science 2002, 297, 967-973; d) Lammelar structures: M. Sofos, J. Goldberger, D. A. Stone, J. E. Allen, Q. Ma, D. J. Herman, W.-W Tsai, L. J. Lauhon, S. I. A. Stupp, Nat. Mater. 2009, 8, 68-75.
[13] a) V. H. O. Wirth, F. U. Herrmann, W. Kern, Macromol. Chem. Phys. 1964, 80, 120-140; b) A. J. Berresheim, M. Müller, K. Müllen, Chem. Rev. 1999, 99, 1747-1786; c) C. Li, M. Liu, N. G. Pschirer, M. Baumgarten, K. Müllen, Chem. Rev. 2010, 110, 6817-6855; d) J. M. Tour, Adv. Mater. 1994, 6, 190-198; e) S. T. Pasco, G. L. Baker, Synth. Met. 1997, 84, 275-276; f) E. A. Weiss, M. J. Ahrens, L. E. Sinks, A. V. Gusev, M. A. Ratner, M. R. Wasielewski, J. Am. Chem. Soc. 2004, 126, 5577-5584; g) E. A. Weiss, M. J. Tauber, R. F. Kelley, M. J. Ahrens, M. A. Ratner, M. R. Wasielewski, J. Am. Chem. Soc. 2005, 127, 11842-11850; h) N. I. Nijegorodov, W. S. Downey, M. B. Danailov, Spectrochim. Acta, Part A 2000, 56, 783-795.
[14] D. Hanss, O, S. Wenger, Eur. J. Inorg. Chem. 2009, 2009, 3778-3790.
[15] a) E. Lörtscher, M. Elbing, M. Tschudy, C. v. Hänisch, H. B. Weber, M. Mayor, H. Riel, ChemPhysChem 2008, 9, 2252-2258; b) D. Hanss, O, S. Wenger, Inorg. Chem. 2008, 47, 9081-9084.
[16] a) D. Hanss, O, S. Wenger, Inorg. Chem. 2009, 48, 671-680; b) M. E. Walther, O, S. Wenger, ChemPhysChem 2009, 10, 1203-1206; c) D. Hanss, M. E. Walther, O, S. Wenger, Chem. Commun. 2010, 46, 7034-7036; d) H. Zhao, J. Liao, J. Ning, Y. Xie, Y. Cao, L. Chen, D. Yang, B. Wang, Adv. Synth. Catal. 2010, 352, 3083-3088.
[17] A. I. Kovalev, K. Takeuchi, M. Asai, M. Ueda, A. L. Rusanov, Russ. Chem. Bull. 2004, 53, 1749-1754.
[18] J. Rotzler, H. Gsellinger, M. Neuburger, D. Vonlanthen, D. Häussinger, M. Mayor, Org. Biomol. Chem. 2011, 9, 86-91.
[19] S. M. Losanitsch, Eur. J. Inorg. Chem. 1897, 30, 1917-1926.
[20] S. Grunder, R. Huber, V. Horhoiu, M. T. González, C. Schönenberger, M. Calame, M. Mayor, J. Org. Chem. 2007, 72, 8337-8344.

1. A compound having a structure of formula (I): 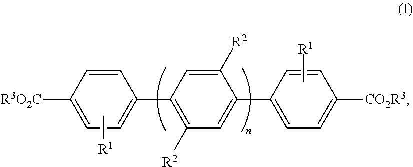
wherein
each R¹ is independently H, OH, alkyl, alkoxy, or amino;
each R² is independently C₁-C₁₂ alkyl, C₁-C₁₂ haloalkyl, nitro, amino, CHO, alkyleneCHO, CN, alkoxy, halo, alkyleneferrocene, —N═NH-aryl, or polyalkyleneoxide;
each R³ is independently H, alkyl, or alkylenearyl, and
n is an integer of 1 to 15.

2. The compound of claim 1, wherein at least one R¹ is OH or R¹ is NH₂, methyl, ethyl, methoxy, or ethoxy.

3. (canceled)

4. The compound of claim 1, wherein at least one R¹ is meta or ortho to —CO₂R³.

5. (canceled)

6. The compound of claim 1, wherein at least one R³ is OH or R³ is methyl or ethyl.

7. (canceled)

8. The compound of claim 1, wherein each R² is selected from the group consisting of methyl, ethyl, hexyl, —O(CH₂CH₂O)ₘCH₃, —N═NHPh, NO₂, and CF₃, and m is an integer of 1 to 20.

9. (canceled)

10. The compound of claim 1, wherein n is 1, 2, 3, 4, 5, 6, 7, 8, 9, 10, 11, 12, 13, 14, or 15.

11.-25. (canceled)

26. The compound of claim 1, wherein no more than two R² are other than methyl.

27. The compound of claim 1, wherein at least one R² is hexyl, —O(CH₂CH₂O)ₘCH₃ and m is an integer of 1 to 10, or —(CH₂)₂ferrocene.

28.-30. (canceled)

31. The compound of claim 1, wherein no more than six R² are other than methyl.

32. A compound selected from

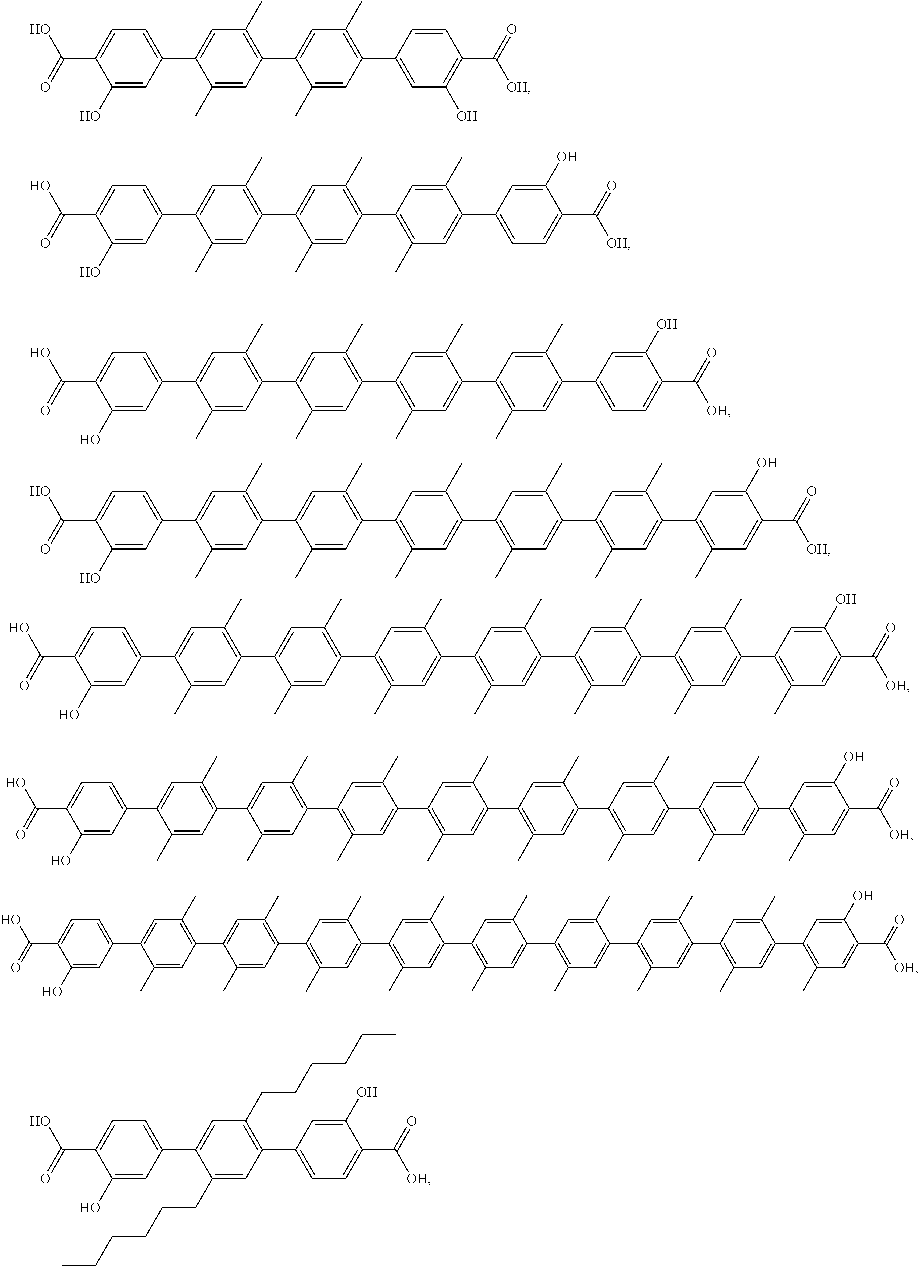

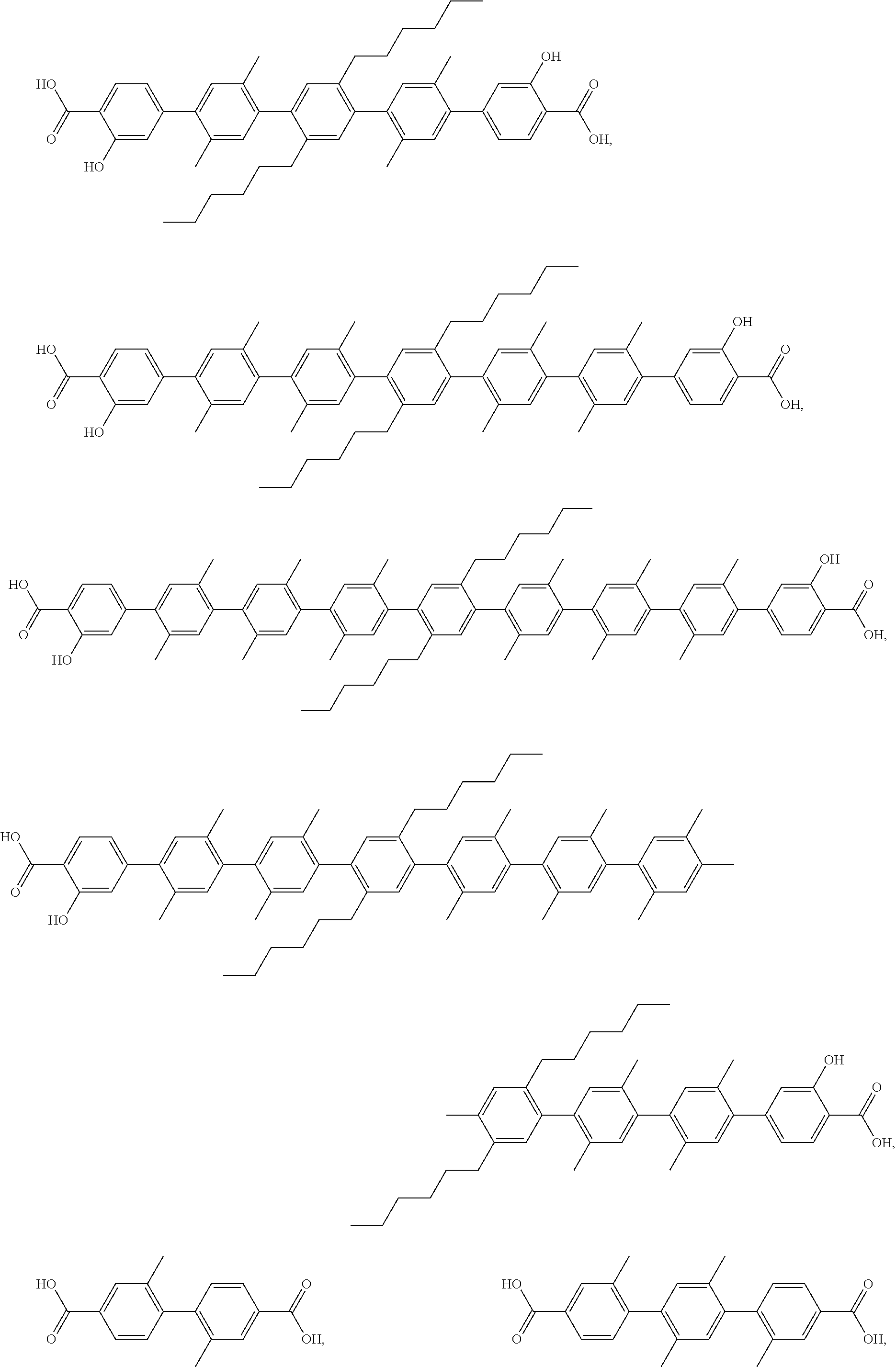

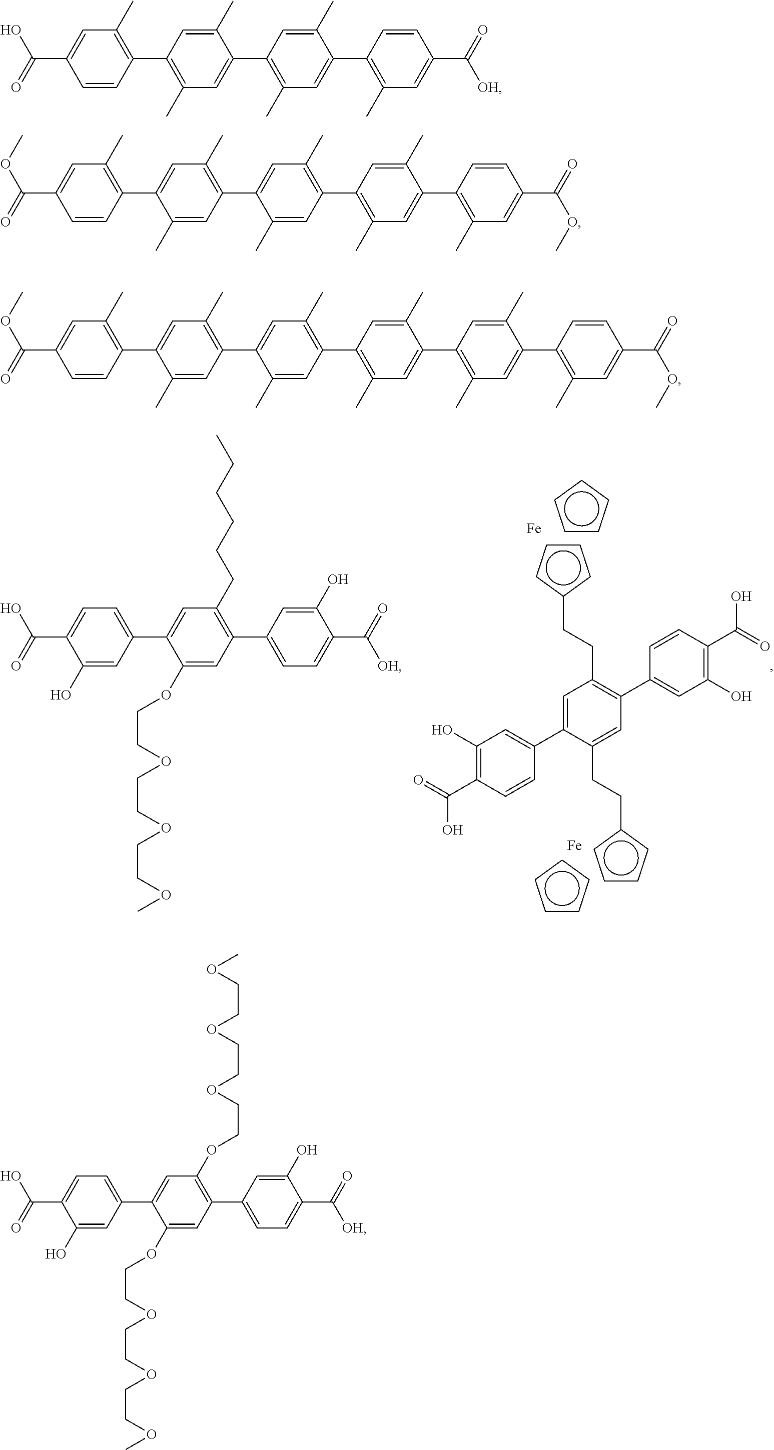

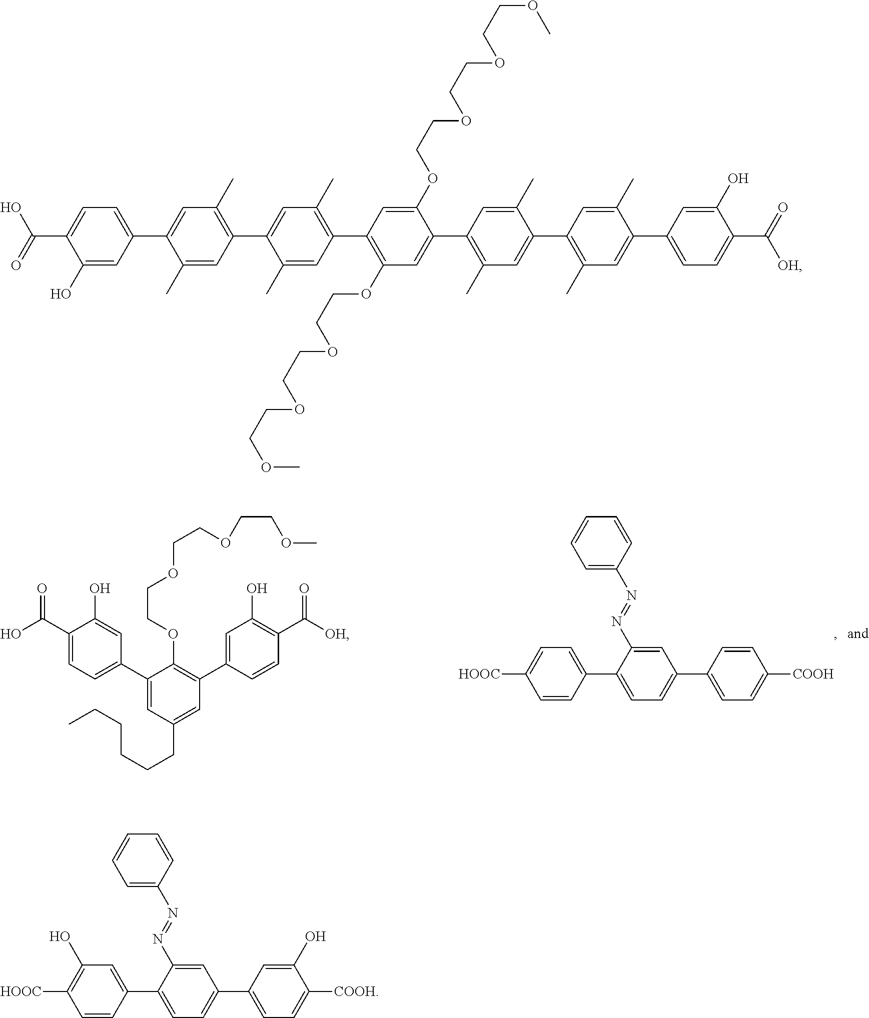 [SMILES: COCCOCCOCCOc1cc(-c2cc(C)c(-c3cc(C)c(-c4ccc(C(=O)O)c(O)c4)cc3C)cc2C)c(OCCOCCOCCOC)cc1-c1cc(C)c(-c2cc(C)c(-c3ccc(C(=O)O)c(O)c3)cc2C)cc1C ; CCCCCCc1cc(-c2ccc(C(=O)O)c(O)c2)c(OCCOCCOCCOC)c(-c2ccc(C(=O)O)c(O)c2)c1 ; O=C(O)c1ccc(-c2ccc(-c3ccc(C(=O)O)cc3)c(/N=N/c3ccccc3)c2)cc1 ; O=C(O)c1ccc(-c2ccc(-c3ccc(C(=O)O)c(O)c3)c(/N=N/c3ccccc3)c2)cc1O]

33. The compound of claim 1 in the form of a nanofiber.

34. The compound of claim 33, wherein the fiber has a length of about 0.5 to about 100 nm.

35. The compound of claim 34, wherein the fiber has a length of about 2 to about 50 nm.

36. The compound of claim 1, in the form of a micro-sphere.

37. The compound of claim 1, in the form of a gel.

38. The compound of claim 37, wherein the gel is formed in an organic solvent.

39. The compound of claim 38, wherein the organic solvent comprises DMSO.

40. The compound of claim 38, wherein the compound has a concentration of about 10 mM to about 1 M in the gel.

41. The compound of claim 40, wherein the concentration is about 10 nM to about 50 mM.
